(19) United States
(12) Patent Application Publication
(10) Pub. No.: US 2008/0096981 A1
(43) Pub. Date: Apr. 24, 2008

(54) Antibodies against inducible TH2 cell factors

(75) Inventors: Charles Qu Dong, Dresher, PA (US); Roy C. Levitt, Ambler, PA (US); Nicholas C. Nicolaides, Boothwyn, PA (US); Yuhong Zhou, Dresher, PA (US); Jamila Louahed, Etterbeek (BE); W. Lee Maloy, Lansdale, PA (US)

Correspondence Address: MORGAN LEWIS & BOCKIUS LLP, 1111 PENNSYLVANIA AVENUE NW, WASHINGTON, DC, 20004, US

(73) Assignee: Genaera Corporation

(21) Appl. No.: 11/641,774

(22) Filed: Dec. 20, 2006

Related U.S. Application Data
(63) Continuation of application No. 09/959,518, filed on Jul. 8, 2002, now abandoned.
(60) Provisional application No. 60/132,138, filed on May 1, 1999.

(51) Int. Cl. A61K 35/00; A61P 11/06; C07H 21/00; C07K 14/00; C07K 16/00; C07K 7/06; C12P 21/00

(52) U.S. Cl. 514/789; 435/69.1; 530/329; 530/350; 530/387.9; 536/23.5

(57) ABSTRACT

A novel TH2 associated gene that is induced by IL-9 has been identified and isolated, thereby providing a therapeutic target for IL-9 mediated diseases such as atopic allergy and asthma-related disorders. The invention also includes methods for the identification and use of small molecule inhibitors of this gene and its products to treat these disorders, methods for diagnosing susceptibility to, and assessing treatment of atopic allergy or asthma-related disorders by measuring the level of gene expression in biologic samples using antibody specific for this protein. The use of this protein as a therapeutic agent for the treatment of autoimmune diseases is also indicated.

### CROSS-REFERENCE TO RELATED APPLICATION

This application claims priority to U.S. Provisional Application No. 60/132,138 filed May 1, 1999, which is herein incorporated by reference in its entirety. This invention is related to the subject matter of U.S. patent application Ser. No. 09/325,571 filed on Jun. 6, 1999, which is herein incorporated by reference.

This application is also related to U.S. patent application Ser. No. 08/980,872 filed on Dec. 1, 1997 and U.S. Provisional Patent Application No. 60/076,815 filed on Mar. 3, 1998, both of which are herein incorporated by reference.

### FIELD OF THE INVENTION

This invention relates to modulating activities associated with the IL-9 pathway for the treatment of atopic allergies and related disorders like asthma. This invention also relates to the use of a novel polypeptide for the treatment of autoimmune diseases.

### BACKGROUND OF THE INVENTION

Inflammation is a complex process in which the body's defense system combats foreign entities. While the battle against foreign entities may be necessary for the body's survival, some defense systems respond to foreign entities, even innocuous ones, as dangerous and thereby damage surrounding tissue in the ensuing battle.

Atopic allergy is an ecogenetic disorder, where genetic background dictates the response to environmental stimuli. The disorder is generally characterized by an increased ability of lymphocytes to produce IgE antibodies in response to ubiquitous antigens. Activation of the immune system by these antigens leads to allergic inflammation and may occur after ingestion, penetration through the skin or after inhalation. When this immune activation occurs and is accompanied by pulmonary inflammation and bronchial hyperresponsiveness, this disorder is broadly characterized as asthma. Certain cells are important in this inflammatory reaction and they include T cells and antigen-presenting cells, B cells that produce IgE, basophils that bind IgE and eosinophils. These inflammatory cells accumulate at the site of allergic inflammation and the toxic products they release contribute to tissue destruction related to these disorders.

While asthma is generally defined as an inflammatory disorder of the airways, clinical symptoms arise from intermittent air flow obstruction. It is a chronic, disabling disorder that appears to be increasing in prevalence and severity (Gergen et al., (1992) Am. Rev. Respir. Dis. 146, 823-824). It is estimated that 30-40% of the population suffer with atopic allergy and 15% of children and 5% of adults in the population suffer from asthma severity (Gergen et al., (1992) Am. Rev. Respir. Dis. 146, 823-824). Thus, an enormous burden is placed on health-care resources.

While most individuals experience similar environmental exposures, only certain individuals develop atopic allergy and asthma. This hypersensitivity to environmental allergens known as “atopy” is often indicated by elevated serum IgE levels or abnormally intense skin test response to allergens in atopic individuals as compared to nonatopics (Marsh et al., (1982) New Eng. J. Med. 305, 1551-1559). Strong evidence for a close relationship between atopic allergy and asthma is derived from the fact that most asthmatics have clinical and serologic evidence of atopy (Clifford et al., (1987) Arch. Dis. Childhood 62, 66-73; Gergen, (1991) Arch. Intern. Med. 151. 487-492; Burrows et al., (1992) J. Allergy Clin. Immunol. 90, 376-385; Johannson et al., (1972) Prog. Clin. Immunol. 1, 1-25; Sears et al., (1991) New Engl. J. Med. 325, 1067-1071; Halonen et al., (1992) Am. Rev. Respir. Dis. 16, 666-670). In particular, younger asthmatics have a high incidence atopy (Marsh et al., (1982) New Eng. J. Med. 305, 1551-1559). In addition, immunologic factors associated with an increase in total serum IgE levels are very closely related to impaired pulmonary function (Burrows et al., (1989) New Eng. J. Med. 320, 271-277). Both the diagnosis and treatment of these disorders are problematic (Gergen et al., (1992) Am. Rev. Respir. Dis. 146, 823-824). The assessment of inflamed lung tissue is often difficult and frequently the source of the inflammation cannot be determined. It is now generally accepted that failure to control pulmonary inflammation leads to significant loss of lung function over time.

Current treatments suffer their own set of disadvantages. The main therapeutic agents, β-agonists, reduce the symptoms thereby transiently improving pulmonary function, but do not affect the underlying inflammation so that lung tissue remains in jeopardy. In addition, constant use of agonists results in desensitization which reduces their efficacy and safety (Molinoff et al., (1995) Goodman and Gilman's The Pharmacologic Basis of Therapeutics, MacMillan Publishing). The agents that can diminish the underlying inflammation, anti-inflammatory steroids, have their own list of disadvantages that range from immunosuppression to bone loss (Molinoff et al., (1995) Goodman and Gilman's The Pharmacologic Basis of Therapeutics, MacMillan Publishing).

Because of the problems associated with conventional therapies, alternative treatment strategies have been evaluated. Glycophorin A (Chu et al., (1992) Cell. Immunol. 145, 223-223), cyclosporin (Alexander et al., (1992) Lancet 339, 324-328; Morely, (1992) Autoimmun. 5 Suppl-A, 265-269) and a nonapeptide fragment of interleukin 2 (IL-2) (Zavyalov et al., (1992) Immunol. Lett. 31, 285-288) all inhibit potentially critical immune functions associated with homeostasis. What is needed in the art is a treatment for asthma that addresses the underlying pathogenesis. Moreover, these therapies must address the episodic nature of the disorder and the close association with allergy and intervene at a point downstream from critical immune functions.

In the related patent applications mentioned above, applicants have demonstrated that interleukin 9 (IL-9), its receptor and activities effected by IL-9 are the appropriate targets for therapeutic intervention in atopic allergy, asthma and related disorders.

Mediator release from mast cells by allergen has long been considered a critical initiating event in allergy. IL-9 was originally identified as a mast cell growth factor and it has been demonstrated that IL-9 up-regulates the expression of mast cell proteases including MCP-1, MCP-2, MCP-4 (Eklund et al., (1993) J. Immunol. 151, 4266-4273) and Granzyme B (Louahed et al., (1995) J. Immunol. 154, 5061-5070). Thus, IL-9 appears to serve a role in the proliferation and differentiation of mast cells. Moreover, IL-9 up-regulates the expression of the alpha chain of the high affinity IgE receptor (Dugas et al., (1993) Eur. J. Immunol. 23, 1687-1692). Elevated IgE levels are considered to be a hallmark of atopic allergy and a risk factor for asthma. Furthermore, both in vitro and in vivo studies have shown IL-9 to potentiate the release of IgE from primed B cells (Petit-Frere et al., (1993) Immunology 79, 146-151).

Based on the data presented in the related patents listed above, there is substantial support for the IL-9 gene candidate in asthma. First, applicants demonstrate linkage, homology between humans and mice, suggesting the same gene is responsible for producing biologic variability in response to antigen in both species. Second, differences in expression of the murine IL-9 candidate gene are associated with biologic variability in bronchial responsiveness. In particular, reduced expression of IL-9 is associated with a lower baseline bronchial response in B6 mice. Third, recent evidence for linkage disequilibrium in data from humans suggests IL-9 may be associated with atopy and bronchial hyperresponsiveness consistent with a role for this gene in both species (Doull et al., (1996) Am. J. Respir. Crit. Care Med. 153, 1280-1284). Moreover, applicants have demonstrated that a genetic alteration in the human gene appears to be associated with loss of cytokine function and lower IgE levels. Fourth, the pleiotropic functions of this cytokine and its receptor in the allergic immune response strongly support a role for the IL-9 pathway in the complex pathogenesis of asthma. Fifth, in humans, biologic variability in the IL-9 receptor also appears to be associated with atopic allergy and asthma. Finally, despite the inherited loss of IL-9 receptor function, these individuals appear to be otherwise healthy. Thus, nature has demonstrated in atopic individuals that the therapeutic down-regulation of IL-9 and IL-9 receptor genes or genes activated by IL-9 and its receptor is likely to be safe.

Thus, the art now understands how the IL-9 gene, its receptor and their functions are related to atopic allergy, asthma and related disorders. Therefore, a specific need in the art exists for elucidation of the role of genes which are regulated by IL-9 in the etiology of these disorders. Furthermore, most significantly, based on this knowledge, there is a need for the identification of agents that are capable of regulating the activity of these genes or their gene products for treating these disorders.

### SUMMARY OF THE INVENTION

Applicants have identified new genes that are tightly expressed in association with an inflammatory response in the airways mediated by type 2 helper T-cells (TH). These genes have been designated TH2AF1. Five murine isotypes have been identified in various strains and tissues (SEQ ID NO: 1, 3, 5, 7, 9) and while one human isotype (SEQ ID NO: 11) has been identified. They are selectively up-regulated by IL-9 and therefore part of the IL-9 signaling pathway. Applicants also claim the polypeptide products of these genes in the mouse (SEQ ID NO: 2, 4, 6, 8, 10) and human (SEQ ID NO: 12). Applicants have satisfied the need for diagnosis and treatment of atopic allergy, asthma and related disorders by demonstrating the role of TH2AF1 in the pathogenesis of these disorders. Therapies for these disorders are derived from the down-regulation of TH2AF1 as a member of the IL-9 pathway.

The identification of TH2AF1 has led to the discovery of agents that are capable of down-regulating its activity. Molecules that down-regulate TH2 AF1 are therefore claimed in the invention. Down-regulation is defined here as a decrease in activation, function or synthesis of TH2AF1, its receptor(s) or activators. It is further defined to include an increase in the degradation of TH2 AF1 gene, its polypeptide product, receptor(s) or activators. Down-regulation is therefore achieved in a number of ways. For example, administration of molecules that can destabilize the binding of TH2AF1 with its receptors(s). Such molecules encompass polypeptide products, including those encoded by the DNA sequences of the TH2AF1 gene or DNA sequences containing various mutations. These mutations may be point mutations, insertions, deletions or spliced variants of the TH2AF1 gene. This invention also includes truncated polypeptides encoded by the DNA molecules described above. These polypeptides being capable of interfering with interaction of TH2AF1 with its receptor and other polypeptides.

A further embodiment of this invention includes the down-regulation of TH2AF1 function by altering expression of the TH2AF1 gene, the use of antisense gene therapy being an example. Down-regulation of TH2AF1 expression is accomplished by administering an effective amount of antisense oligonucleotides. These antisense molecules can be fashioned from the DNA sequence of the TH2AF1 gene or sequences containing various mutations, deletions, insertions or spliced variants. Another embodiment of this invention relates to the use of isolated RNA or DNA sequences derived from the TH2AF1 gene. These sequences containing various mutations such as point mutations, insertions, deletions or spliced variant mutations of TH2AF1 gene and can be useful in gene therapy.

Molecules that increase the degradation of the TH2AF1 polypeptide may also be used to down-regulate its functions and are within the scope of the invention. Phosphorylation of TH2AF1 may alter protein stability, therefore kinase inhibitors may be used to down-regulate its function. Glycosylation of TH2AF1 may alter protein stability, therefore glycosylase inhibitors may be used to down-regulate its function. Down-regulation of TH2AF1 may also be accomplished by the use of polyclonal or monoclonal antibodies or fragments thereof directed against the TH2AF1 polypeptide. Such molecules are within the claimed invention. This invention further includes small molecules with the three-dimensional structure necessary to bind with sufficient affinity to block TH2AF1 interactions with its receptor(s). In a further embodiment, aminosterol agents are assessed for their ability to block TH2AF1 induction by IL-9 or antigen as a means of determining their usefulness in treating atopic allergies and related disorders.

The products discussed above represent various effective therapeutic agents in treating atopic allergies, asthma and other related disorders. Applicants have thus provided antagonists and methods of identifying antagonists that are capable of down-regulating TH2AF1. Applicants also provide methods for down-regulating the activity of TH2AF1 by administering truncated polypeptide products, aminosterols and the like.

Applicants also provide a method for the diagnosis of susceptibility to atopic allergy, asthma and related disorders by describing a method for assaying the induction of TH2AF1, its functions or downstream activities. In a further embodiment, applicants provide methods to monitor the effects of TH2 AF1 down-regulation as a means to follow the treatment of atopic allergy and asthma.

Applicants also provide a method for the treatment of autoimmune diseases such as inflammatory bowel disease (IBD), which have been previously shown to be treatable with the use of TH2-type polypeptides (Del Prete, (1998) Int. Rev. Immunol., 16, 427-455). The application of TH2AF1 as a pharmacologic agent for the treatment of autoimmune diseases is suggested in part by its TH2-associated expression profile, and its induction by the cytokine interleukin-10, a TH2-type protein previously shown to have suppressive activity in IBD models (Opal et al., Clin. Infect. Dis., 27, 1497-507).

The accompanying figures, which are incorporated in and constitute a part of this specification, illustrate several embodiments of the invention and together with the description, serve to explain the principle of the invention.

### BRIEF DESCRIPTION OF THE FIGURES

FIG. 1—Schematic diagram of the suppressive PCR cDNA subtraction technique.

FIG. 2—Alignment of murine TH2AF1 isoforms derived from the TG5 intestine (TH2AF1.c), the FVB small intestine (TH2AF1.s) and the TG5 and FVB lung (TH2AF1.1).

FIG. 3—TH2AF1 expression in the lung of normal mice (FVB) compared to IL-9 transgenic mice (TG5). GADPH is used as an internal control.

FIG. 4—Expression of TH2 AF1 in the lungs of DBA (M) and C57BL/6 (B6) mice. RNA loading is shown on bottom panel.

FIG. 5—Expression of TH2AF1 in the lung of the C57BL/6 mouse with and without intratracheal administration of recombinant cytokines. RNA loading is shown on bottom panel.

FIG. 6—Expression of TH2AF1 in the lung of antigen exposed TG5 (T) and FVB (F) mice.

FIG. 7—Production of antibodies capable of detecting both the mouse and human TH2AF1 proteins. Anti-TH2AF1 (6873a and 6874a) recognize murine and human Flag-tagged proteins. TNT panel shows starting material from in vitro translation reactions. IgG panel shows immunoprecipitation (IP) using rabbit preimmune antiserum. Flag panel shows IP of Flag-tagged TH2AF1 proteins. Panels 6873 and 6874 show IP of TH2AF1 proteins with anti-TH2AF1 antiserum. Lane 1: unprogrammed lysates; Lane 2: lysate programmed with murine TH2AF1-Flag template; Lane 3: lysate programmed with human TH2 AF1-Flag template; Lane 4: lysate programmed with human IL-9 receptor as negative control.

FIG. 8—Down regulation of TH2AF1 by small molecule weight molecules in antigen models of airway inflammation. GADPH is used as an internal control.

FIG. 9—Demonstration of TH2AF1 protein secretion by western blot.

FIG. 10—TH2AF can activate human lymphocytes in vitro.

FIG. 11—Recombinant production of TH2AF1 in yeast cells.

FIG. 12—Recombinant TH2 AF1 has mitogenic activity on human primary lymphocytes. Splenocytes were plated with or without PRA or TH2AF1 and grown for forty-eight hours. Lymphocyte activation was determined by the number of cellular aggregates (arrows).

FIG. 13—TH2AF1 found in the lung of asthmatics.

### DETAILED DESCRIPTION OF THE PREFERRED EMBODIMENT

### TH2 AF1 Proteins

The present invention provides isolated protein, allelic variants of the protein, and conservative amino acid substitutions of the TH2AF1 family of proteins. As used herein, the protein or polypeptide refers to a protein that has the amino acid sequence depicted in SEQ ID NO: 2, 4, 6, 8, 10 or 12. The invention also includes naturally occurring allelic variants and proteins that have a slightly different amino acid sequence than that specifically recited above. Allelic variants, though possessing a slightly different amino acid sequence than those recited above, will still have the same or similar biological functions associated with the TH2AF1 protein.

As used herein, the family of proteins related to the TH2AF1 protein refers to proteins that have been isolated from organisms in addition to humans or mice. The methods used to identify and isolate other members of the family of proteins related to the TH2AF1 protein are described below.

The proteins of the present invention are preferably in isolated form. As used herein, a protein is said to be isolated when physical, mechanical or chemical methods are employed to remove the protein from cellular constituents that are normally associated with the protein. A skilled artisan can readily employ standard purification methods to obtain an isolated protein.

The proteins of the present invention further include conservative variants of the proteins herein described. As used herein, a conservative variant refers to alterations in the amino acid sequence that do not adversely affect the biological functions of the protein. A substitution, insertion or deletion is said to adversely affect the protein when the altered sequence prevents or disrupts a biological function associated with the protein. For example, the overall charge, structure or hydrophobic and hydrophilic properties of the protein can be altered without adversely affecting a biological activity. Accordingly, the amino acid sequence can be altered, for example to render the peptide more hydrophobic or hydrophilic, without adversely affecting the biological activities of the protein.

Ordinarily, the allelic variants, the conservative substitution variants, and the members of the protein family, will have an amino acid sequence having at least seventy-two percent or seventy-five percent amino acid sequence identity with the sequence set forth in SEQ ID NO: 2, 4, 6, 8, 10 or 12, more preferably at least eighty percent, even more preferably at least ninety percent, and most preferably at least ninety-five percent. Identity or homology with respect to such sequences is defined herein as the percentage of amino acid residues in the candidate sequence that are identical with the known peptides, after aligning the sequences and introducing gaps, if necessary, to achieve the maximum percent homology, and not considering any conservative substitutions as part of the sequence identity. N-terminal, C-terminal or internal extensions, deletions, or insertions into the peptide sequence shall not be construed as affecting homology.

Thus, the proteins of the present invention include molecules having the amino acid sequence disclosed in SEQ ID NO: 2, 4, 6, 8, 10 or 12; fragments thereof having a consecutive sequence of at least about 3, 4, 5, 6, 10, 15, 20, 25, 30, 35 or more amino acid residues of the TH2AF1 protein; amino acid sequence variants of such sequence wherein at least one amino acid residue has been inserted N- or C-terminal to, or within, the disclosed sequence; amino acid sequence variants of the disclosed sequence, or their fragments as defined above, that have been substituted by another residue. Contemplated variants further include those containing predetermined mutations by, e.g., homologous recombination, site-directed or PCR mutagenesis, and the corresponding proteins of other animal species, including but not limited to rabbit, rat, porcine, bovine, ovine, equine and non-human primate species, the alleles or other naturally occurring variants of the family of proteins; and derivatives wherein the protein has been covalently modified by substitution, chemical, enzymatic, or other appropriate means with a moiety other than a naturally occurring amino acid (for example, a detectable moiety such as an enzyme or radioisotope).

As described below, members of the family of proteins can be used: (1) to identify agents which modulate at least one activity of the protein, (2) in methods of identifying binding partners for the protein, (3) as an antigen to raise polyclonal or monoclonal antibodies, and 4) as a therapeutic agent in the treatment of asthma and asthma-related disorders.

TH2AF1 Nucleic Acids

The present invention further provides nucleic acid molecules that encode the protein having SEQ ID NO: 2, 4, 6, 8, 10 or 12 and the related proteins herein described, preferably in isolated form. As used herein, “nucleic acid” is defined as RNA or DNA that encodes a protein or peptide as defined above, or is complementary to nucleic acid sequence encoding such peptides, or hybridizes to such nucleic acid and remains stably bound to it under appropriate stringency conditions, or encodes a polypeptide sharing at least seventy-two percent or seventy-five percent sequence identity, preferably at least eighty percent, and more preferably at least eighty-five percent, with the peptide sequences. Specifically contemplated are genomic DNA, cDNA, mRNA and antisense molecules, as well as nucleic acids based on alternative backbones or including alternative bases whether derived from natural sources or synthesized. Such hybridizing or complementary nucleic acids, however, are defined further as being novel and nonobvious over any prior art nucleic acid including that which encodes, hybridizes under appropriate stringency conditions, or is complementary to nucleic acid encoding a protein according to the present invention.

Homology or identity is determined by BLAST (Basic Local Alignment Search Tool) analysis using the algorithm employed by the programs blastp, blastn, blastx, tblastn and tblastx (Karlin et al., (1990) Proc. Natl. Acad. Sci. USA 87, 2264-2268; Altschul, (1993) J. Mol. Evol. 36, 290-300, fully incorporated by reference) which are tailored for sequence similarity searching. The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al., (Nature Genetics (1994) 6, 119-129) which is fully incorporated by reference. The search parameters for histogram, descriptions, alignments, expect (i.e., the statistical significance threshold for reporting matches against database sequences), cutoff, matrix and filter are at the default settings. The default scoring matrix used by BLASTp, BLASTx, tBLASTn, and tBLASTx is the BLOSUM62 matrix (Henikoff et al, (1992) Proc. Natl. Acad. Sci. USA 89, 10915-10919, fully incorporated by reference). Four BLASTn parameters were adjusted as follows: Q=10 (gap creation penalty); R=10 (gap extension penalty); wink=1 (generates word hits at every winks position along the query); and gapw=16 (sets the window width within which gapped alignments are generated). The equivalent BLASTp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

“Stringent conditions” are those that (1) employ low ionic strength and high temperature for washing, for example, 0.015 M NaCl/0.0015 M sodium citrate/0.1% SDS at 50° C. or (2) employ during hybridization a denaturing agent such as formamide, for example, 50% formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is use of 50% formamide, 5×SSC, 50 mM sodium phosphate (pH 6.8), 0.1% sodium pyrophosphate, 5×Denhardt's solution, sonicated salmon sperm DNA (50 μg/ml), 0.1% SDS and 10% dextran sulfate at 42° C., with washes at 42° C. in 0.2×SSC and 0.1% SDS. A skilled artisan can readily determine and vary the stringency conditions appropriately to obtain a clear and detectable hybridization signal.

As used herein, a nucleic acid molecule is said to be “isolated” when the nucleic acid molecule is substantially separated from contaminant nucleic acid encoding other polypeptides from the source of nucleic acid.

The present invention further provides fragments of the encoding nucleic acid molecule. As used herein, a fragment of an encoding nucleic acid molecule refers to a small portion of the entire protein encoding sequence. The size of the fragment will be determined by the intended use. For example, if the fragment is chosen so as to encode an active portion of the protein, the fragment will need to be large enough to encode the functional region(s) of the protein. If the fragment is to be used as a nucleic acid probe or PCR primer, then the fragment length is chosen so as to obtain a relatively small number of false positives during probing or priming.

Fragments of the encoding nucleic acid molecules of the present invention (i.e., synthetic oligonucleotides) that are used as probes or specific primers for the polymerase chain reaction (PCR) or to synthesize gene sequences encoding proteins of the invention can easily be synthesized by chemical techniques, for example, the phosphotriester method of Matteucci et al., (1981) (J. Am. Chem. Soc. 103, 3185-3191) or using automated synthesis methods. In addition, larger DNA segments can readily be prepared by well known methods, such as synthesis of a group of oligonucleotides that define various modular segments of the gene, followed by ligation of oligonucleotides to build the complete modified gene.

The encoding nucleic acid molecules of the present invention may further be modified so as to contain a detectable label for diagnostic and probe purposes. A variety of such labels are known in the art and can readily be employed with the encoding molecules herein described. Suitable labels include, but are not limited to, biotin, radiolabeled nucleotides and the like. A skilled artisan can employ any of the art known labels to obtain a labeled encoding nucleic acid molecule.

Modifications to the primary structure itself by deletion, addition, or alteration of the amino acids incorporated into the protein sequence during translation can be made without destroying the activity of the protein. Such substitutions or other alterations result in proteins having an amino acid sequence encoded by a nucleic acid falling within the contemplated scope of the present invention.

As described above, the identification of the nucleic acid molecule having SEQ ID NO: 1, 3, 5, 7 or 9 in mice and SEQ ID NO: 11 in humans allows a skilled artisan to isolate nucleic acid molecules that encode other members of the protein family in addition to the murine and human sequences herein described. Further, the presently disclosed nucleic acid molecules allow a skilled artisan to isolate nucleic acid molecules that encode other members of the family of proteins in addition to the TH2AF1 protein having SEQ ID NO: 2, 4, 6, 8, 10 or 12.

Essentially, a skilled artisan can readily use the amino acid sequence of SEQ ID NO: 2, 4, 6, 8, 10 or 12 to generate antibody probes to screen expression libraries prepared from appropriate cells. Typically, polyclonal antiserum from mammals such as rabbits immunized with the purified protein (as described below) or monoclonal antibodies can be used to probe a mammalian cDNA or genomic expression library, such as lambda gtll library, to obtain the appropriate coding sequence for other members of the protein family. The cloned cDNA sequence can be expressed as a fusion protein, expressed directly using its own control sequences, or expressed by constructions using control sequences appropriate to the particular host used for expression of the enzyme.

Alternatively, a portion of the coding sequence herein described can be synthesized and used as a probe to retrieve DNA encoding a member of the protein family from any mammalian organism. Oligomers containing approximately 18-20 nucleotides (encoding about a six to seven amino acid stretch) are prepared and used to screen genomic DNA or cDNA libraries to obtain hybridization under stringent conditions or conditions of sufficient stringency to eliminate an undue level of false positives.

Additionally, pairs of oligonucleotide primers can be prepared for use in a polymerase chain reaction (PCR) to selectively clone an encoding nucleic acid molecule. A PCR denature/anneal/extend cycle for using such PCR primers is well known in the art and can readily be adapted for use in isolating other encoding nucleic acid molecules.

Vectors

The present invention further provides recombinant DNA molecules (rDNA) that contain a coding sequence. As used herein, a rDNA molecule is a DNA molecule that has been subjected to molecular manipulation. Methods for generating rDNA molecules are well known in the art, for example, see Sambrook et al., (1985) Molecular Cloning—A Laboratory Manual, Cold Spring Harbor Laboratory Press. In the preferred rDNA molecules, a coding DNA sequence is operably linked to expression control sequences and/or vector sequences.

The choice of vector and expression control sequences to which one of the protein family encoding sequences of the present invention is operably linked depends directly, as is well known in the art, on the functional properties desired (e.g., protein expression, and the host cell to be transformed). A vector contemplated by the present invention is at least capable of directing the replication or insertion into the host chromosome, and preferably also expression, of the structural gene included in the rDNA molecule.

Expression control elements that are used for regulating the expression of an operably linked protein encoding sequence are known in the art and include, but are not limited to, inducible promoters, constitutive promoters, secretion signals, and other regulatory elements. Preferably, the inducible promoter is readily controlled, such as being responsive to a nutrient in the host cell's medium.

In one embodiment, the vector containing a coding nucleic acid molecule will include a prokaryotic replicon, i.e., a DNA sequence having the ability to direct autonomous replication and maintenance of the recombinant DNA molecule extra-chromosomally in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical bacterial drug resistance genes are those that confer resistance to ampicillin or tetracycline.

Vectors that include a prokaryotic replicon can further include a prokaryotic or bacteriophage promoter capable of directing the expression (transcription and translation) of the coding gene sequences in a bacterial host cell, such as E. coli. A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment of the present invention. Typical of such vector plasmids are pUC8, pUC9, pBR322 and pBR329 (Biorad Laboratories), pPL and pKK223 (Pharmacia).

Expression vectors compatible with eukaryotic cells, preferably those compatible with vertebrate cells, can also be used to form a rDNA molecules that contains a coding sequence. Eukaryotic cell expression vectors are well known in the art and are available from several commercial sources. Typically, such vectors are provided containing convenient restriction sites for insertion of the desired DNA segment. Typical of such vectors are pSVL and pKSV-10 (Pharmacia), pBPV-1, pML2d (International Biotechnologies), pTDT1 (ATCC, #31255) and the like eukaryotic expression vectors.

Eukaryotic cell expression vectors used to construct the rDNA molecules of the present invention may further include a selectable marker that is effective in an eukaryotic cell, preferably a drug resistance selection marker. A preferred drug resistance marker is the gene whose expression results in neomycin resistance, i.e., the neomycin phosphotransferase (neo) gene. (Southern et al., (1982) J. Mol. Anal. Genet. 1, 327-341). Alternatively, the selectable marker can be present on a separate plasmid, the two vectors introduced by co-transfection of the host cell, and transfectants selected by culturing in the appropriate drug for the selectable marker.

The present invention further provides host cells transformed with a nucleic acid molecule that encodes a protein of the present invention. The host cell can be either prokaryotic or eukaryotic. Eukaryotic cells useful for expression of a protein of the invention are not limited, so long as the cell line is compatible with cell culture methods and compatible with the propagation of the expression vector and expression of the gene product. Preferred eukaryotic host cells include, but are not limited to, yeast, insect and mammalian cells, preferably vertebrate cells such as those from a mouse, rat, monkey or human cell line. Preferred eukaryotic host cells include Chinese hamster ovary (CHO) cells available from the ATCC as CCL61, NIH Swiss mouse embryo cells NIH-3T3 available from the ATCC as CRL1658, baby hamster kidney cells (BHK), and the like eukaryotic tissue culture cell lines.

Any prokaryotic host can be used to express a rDNA molecule encoding a protein of the invention. The preferred prokaryotic hosts include, but are not limited to, Pichia and Saccromyces.

Transformation of appropriate cell hosts with a rDNA molecule of the present invention is accomplished by well known methods that typically depend on the type of vector used and host system employed. With regard to transformation of prokaryotic host cells, electroporation and salt treatment methods are typically employed (see, for example, Maniatis et al., (1982) Molecular Cloning—A Laboratory Manual, Cold Spring Harbor Laboratory Press; Cohen et al., (1972) Proc. Natl. Acad. Sci. USA 69, 2110-2114). With regard to transformation of vertebrate cells with vectors containing rDNA, electroporation, cationic lipid or salt treatment methods are typically employed (see, for example, Graham et al., (1973) Virology 52, 456-467; Wigler et al., (1979) Proc. Natl. Acad. Sci. USA 76, 1373-1376).

Successfully transformed cells, i.e., cells that contain a rDNA molecule of the present invention, can be identified by well known techniques including the selection for a selectable marker. For example, cells resulting from the introduction of an rDNA of the present invention can be cloned to produce single colonies. Cells from those colonies can be harvested, lysed and their DNA content examined for the presence of the rDNA using a method such as that described by Southern, (1975) J. Mol. Biol. 98, 503-517 or the proteins produced from the cell assayed via an immunological method.

The present invention further provides methods for producing a protein of the invention using nucleic acid molecules herein described. In general terms, the production of a recombinant form of a protein typically involves the following steps: First, a nucleic acid molecule is obtained that encodes a protein of the invention, such as the nucleic acid molecule comprising SEQ ID NO: 1, 3, 5, 7, 9 or 11, or nucleotides 67-1005 of SEQ ID NO: 1, 3, 5, 7 or 9, or nucleotides 104-1042 of SEQ ID NO: 11. If the encoding sequence is uninterrupted by introns as is the case here, it is directly suitable for expression in any host.

The nucleic acid molecule is then preferably placed in operable linkage with suitable control sequences, as described above, to form an expression unit containing the protein open reading frame. The expression unit is used to transform a suitable host and the transformed host is cultured under conditions that allow the production of the recombinant protein. Optionally the recombinant protein is isolated from the medium or from the cells; recovery and purification of the protein may not be necessary in some instances where some impurities may be tolerated.

Each of the foregoing steps can be done in a variety of ways. For example, the desired coding sequences may be obtained from genomic fragments and used directly in appropriate hosts. The construction of expression vectors that are operable in a variety of hosts is accomplished using appropriate replicons and control sequences, as set forth above. The control sequences, expression vectors, and transformation methods are dependent on the type of host cell used to express the gene and were discussed in detail earlier. Suitable restriction sites can, if not normally available, be added to the ends of the coding sequence so as to provide an excisable gene to insert into these vectors. A skilled artisan can readily adapt any host/expression system known in the art for use with the nucleic acid molecules of the invention to produce recombinant protein.

Assays

Another embodiment of the present invention provides methods for use in isolating and identifying binding partners of proteins of the invention In detail, a protein of the invention is mixed with a potential binding partner or an extract or fraction of a cell under conditions that allow the association of potential binding partners with the protein of the invention. After mixing, peptides, polypeptides, proteins or other molecules that have become associated with a protein of the invention are separated from the mixture. The binding partner bound to the protein of the invention can then be removed and further analyzed. To identify and isolate a binding partner, the entire protein, for instance the entire TH2AF1 protein of SEQ ID NO: 2, 4, 6, 8, 10 or 12 can be used. Alternatively, a fragment of the protein can be used.

As used herein, a cellular extract refers to a preparation or fraction which is made from a lysed or disrupted cell. The preferred source of cellular extracts will be cells derived from human lung tissue, for instance, asthmatic human lung tissue. Alternatively, cellular extracts may be prepared from normal human lung tissue or available cell lines, particularly lung or epithelial derived cell lines. Cellular extracts include those cells isolated from bronchial alveolar lavage (BAL) from both humans and laboratory animals.

A variety of methods can be used to obtain an extract of a cell. Cells can be disrupted using either physical or chemical disruption methods. Examples of physical disruption methods include, but are not limited to, sonication and mechanical shearing. Examples of chemical lysis methods include, but are not limited to, detergent lysis and enzyme lysis. A skilled artisan can readily adapt methods for preparing cellular extracts in order to obtain extracts for use in the present methods. Once an extract of a cell is prepared, the extract is mixed with the protein of the invention under conditions in which association of the protein with the binding partner can occur. A variety of conditions can be used, the most preferred being conditions that closely resemble conditions found in the cytoplasm of a human cell. Features such as osmolarity, pH, temperature, and the concentration of cellular extract used, can be varied to optimize the association of the protein with the binding partner.

After mixing under appropriate conditions, the bound complex is separated from the mixture. A variety of techniques can be utilized to separate the mixture. For example, antibodies specific to a protein of the invention can be used to immunoprecipitate the binding partner complex. Alternatively, standard chemical separation techniques such as chromatography and density-sediment centrifugation can be used.

After removal of non-associated cellular constituents found in the extract, the binding partner can be dissociated from the complex using conventional methods. For example, dissociation can be accomplished by altering the salt concentration or pH of the mixture.

To aid in separating associated binding partner pairs from the mixed extract, the protein of the invention can be immobilized on a solid support. For example, the protein can be attached to a nitrocellulose matrix or acrylic beads. Attachment of the protein to a solid support aids in separating peptide-binding partner pairs from other constituents found in the extract. The identified binding partners can be either a single protein or a complex made up of two or more proteins. Alternatively, binding partners may be identified using a Far-Western assay according to the procedures of Takayama et al., (997) Methods Mol. Biol. 69, 171-184 or Sauder et al., (1996) J. Gen. Virol. 77, 991-996 or identified through the use of epitome tagged proteins or GST fusion proteins.

Alternatively, the nucleic acid molecules of the invention can be used in a yeast two-hybrid system. The yeast two-hybrid system has been used to identify other protein partner pairs and can readily be adapted to employ the nucleic acid molecules herein described (Stratagene Hybrizap® two-hybrid system).

Another embodiment of the present invention provides methods for identifying agents that modulate the expression of a nucleic acid encoding a protein of the invention such as a protein having the amino acid sequence of SEQ ID NO: 2, 4, 6, 8, 10 or 12. Such assays may utilize any available means of monitoring for changes in the expression level of the nucleic acids of the invention. As used herein, an agent is said to modulate the expression of a nucleic acid of the invention, for instance a nucleic acid encoding the protein having the sequence of SEQ ID NO: 2, 4, 6, 8, 10 or 12, if it is capable of up- or down-regulating expression of the nucleic acid in a cell. In one assay format, cell lines that contain reporter gene fusions between the open reading frame defined by nucleotides 67-1005 of SEQ ID NO: 1, 3, 5, 7 or 9; or nucleotides 104-1042 of SEQ ID NO: 11, and any assayable fusion partner may be prepared. Numerous assayable fusion partners are known and readily available including the firefly luciferase gene and the gene encoding chloramphenicol acetyltransferase (Alam et al., (1990) Anal. Biochem. 188, 245-254). Cell lines containing the reporter gene fusions are then exposed to the agent to be tested under appropriate conditions and time. Differential expression of the reporter gene between samples exposed to the agent and control samples identifies agents which modulate the expression of a nucleic acid encoding the protein having the sequence of SEQ ID NO: 2, 4, 6, 8, 10 or 12.

Additional assay formats may be used to monitor the ability of the agent to modulate the expression of a nucleic acid encoding a protein of the invention such as the protein having SEQ ID NO: 2, 4, 6, 8, 10 or 12. For instance, mRNA expression may be monitored directly by hybridization to the nucleic acids of the invention. Cell lines are exposed to the agent to be tested under appropriate conditions and time and total RNA or mRNA is isolated by standard procedures such those disclosed in Sambrook et al., (1985) Molecular Cloning—A Laboratory Manual, Cold Spring Harbor Laboratory Press.

Probes to detect differences in RNA expression levels between cells exposed to the agent and control cells may be prepared from the nucleic acids of the invention. It is preferable, but not necessary, to design probes which hybridize only with target nucleic acids under conditions of high stringency. Only highly complementary nucleic acid hybrids form under conditions of high stringency. Accordingly, the stringency of the assay conditions determines the amount of complementarity which should exist between two nucleic acid strands in order to form a hybrid. Stringency should be chosen to maximize the difference in stability between the probe:target hybrid and potential probe:non-target hybrids.

Probes may be designed from the nucleic acids of the invention through methods known in the art. For instance, the G+C content of the probe and the probe length can affect probe binding to its target sequence. Methods to optimize probe specificity are commonly available in Sambrook et al., (1985) Molecular Cloning—A Laboratory Manual, Cold Spring Harbor Laboratory Press or Ausubel et al., (1995) Current Protocols in Molecular Biology, Greene Publishing.

Hybridization conditions are modified using known methods, such as those described by Sambrook et al., (1985) and Ausubel et al., (1995) as required for each probe. Hybridization of total cellular RNA or RNA enriched for polyA+ RNA can be accomplished in any available format. For instance, total cellular RNA or RNA enriched for polyA+ RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a silicon based wafer or a porous glass wafer. The wafer can then be exposed to total cellular RNA or polyA+ RNA from a sample under conditions in which the affixed sequences will specifically hybridize. Such wafers and hybridization methods are widely available, for example, those disclosed by Beattie (WO9511755). By examining for the ability of a given probe to specifically hybridize to a RNA sample from an untreated cell population and from a cell population exposed to the agent, agents which up or down regulate the expression of a nucleic acid encoding the protein having the sequence of SEQ ID NO: 2, 4, 6, 8, 10 or 12 are identified.

Hybridization for qualitative and quantitative analysis of mRNA may also be carried out by using a RNase Protection Assay (i.e., RPA, see Ma et al., (1996) Methods 10, 273-238). Briefly, an expression vehicle comprising cDNA encoding the gene product and a phage specific DNA dependent RNA polymerase promoter (e.g., T7, T3 or SP6 RNA polymerase) is linearized at the 3′ end of the cDNA molecule, downstream from the phage promoter, wherein such a linearized molecule is subsequently used as a template for synthesis of a labeled antisense transcript of the cDNA by in vitro transcription. The labeled transcript is then hybridized to a mixture of isolated RNA (i.e., total or fractionated mRNA) by incubation at 45° C. overnight in a buffer comprising 80% formamide, 40 mM Pipes, pH 6.4, 0.4 M NaCl and 1 mM EDTA. The resulting hybrids are then digested in a buffer comprising 40 μg/ml ribonuclease A and 2 μg/ml ribonuclease: After deactivation and extraction of extraneous proteins, the samples are loaded onto urea/polyacrylamide gels for analysis.

In another assay format, agents which effect the expression of the instant gene products, cells or cell lines would first be identified which express said gene products physiologically. Cells and cell lines so identified would be expected to comprise the necessary cellular machinery such that the fidelity of modulation of the transcriptional apparatus is maintained with regard to exogenous contact of agent with appropriate surface transduction mechanisms and the cytosolic cascades. Further, such cells or cell lines would be transduced or transfected with an expression vehicle (e.g., a plasmid or viral vector) construct comprising an operable non-translated 5′-promoter containing end of the structural gene encoding the instant gene products fused to one or more antigenic fragments, which are peculiar to the instant gene products, wherein said fragments are under the transcriptional control of said promoter and are expressed as polypeptides whose molecular weight can be distinguished from the naturally occurring polypeptides or may further comprise an immunologically distinct tag. Such a process is well known in the art (see, Maniatis et al., (1982) Molecular Cloning—A Laboratory Manual, Cold Spring Harbor Laboratory Press).

Cells or cell lines transduced or transfected as outlined above would then be contacted with agents under appropriate conditions; for example, the agent comprises a pharmaceutically acceptable excipient and is contacted with Cells in an aqueous physiological buffer such as phosphate buffered saline (PBS) at physiological pH, Eagles balanced salt solution (BSS) at physiological pH, PBS or BSS comprising serum or conditioned media comprising PBS or BSS and/or serum incubated at 37 C. Said conditions may be modulated as deemed necessary by one of skill in the art. Subsequent to contacting the cells with the agent, said cells will be disrupted and the polypeptides of the disruptate are fractionated such that a polypeptide fraction is pooled and contacted with an antibody to be further processed by immunological assay (e.g., ELISA, immunoprecipitation or Western blot). The pool of proteins isolated from the “agent contacted” sample will be compared with a control sample where only the excipient is contacted with the cells and an increase or decrease in the immunologically generated signal from the “agent contacted” sample compared to the control will be used to distinguish the effectiveness of the agent.

Another embodiment of the present invention provides methods for identifying agents that modulate at least one activity of a protein of the invention such as the protein having the amino acid sequence of SEQ ID NO: 2, 4, 6, 8, 10 or 12. Such methods or assays may utilize any means of monitoring or detecting the desired activity.

In one format, the relative amounts of a protein of the invention between a cell population that has been exposed to the agent to be tested compared to an unexposed control cell population may be assayed. In this format, probes such as specific antibodies are used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with the probe.

Antibody probes are prepared by immunizing suitable mammalian hosts in appropriate immunization protocols using the peptides. Polypeptides or proteins of the invention if they are of sufficient length, or if desired, or if required to enhance immunogenicity, conjugated to suitable carriers. Methods for preparing immunogenic conjugates with carriers such as BSA, KLH, or other carrier proteins are well known in the art. In some circumstances, direct conjugation using, for example, carbodiimide reagents may be effective; in other instances linking reagents such as those supplied by Pierce Chemical Co. may be desirable to provide accessibility to the hapten. The hapten peptides can be extended at either the amino or carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation.

While the polyclonal antisera produced in this way may be satisfactory for some applications, for pharmaceutical compositions, use of monoclonal preparations is preferred. Immortalized cell lines which secrete the desired monoclonal antibodies may be prepared using the standard method of Kohler & Milstein (Biotechnology (1992) 24, 524-526) or modifications which effect immortalization of lymphocytes or spleen cells, as is generally known. The immortalized cell lines secreting the desired antibodies are screened by immunoassay in which the antigen is the peptide hapten, polypeptide or protein. When the appropriate immortalized cell culture secreting the desired antibody is identified, the cells can be cultured either in vitro or by production in ascites fluid.

The desired monoclonal antibodies may be recovered from the culture supernatant or from the ascites supernatant. Fragments of the monoclonals or the polyclonal antisera which contain the immunologically significant portion can be used as antagonists, as well as the intact antibodies. Use of immunologically reactive fragments, such as the Fab, Fab′ of F(ab)₂ fragments is often preferable, especially in a therapeutic context, as these fragments are generally less immunogenic than the whole immunoglobulin. The antibodies or fragments may also be produced, using current technology, by recombinant means. Antibody regions that bind specifically to the desired regions of the protein can also be produced in the context of chimeras with multiple species origin.

Agents that are assayed in the above method can be randomly selected or rationally selected or designed. As used herein, an agent is said to be randomly selected when the agent is chosen randomly without considering the specific sequences involved in the association of the a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a non-random basis which takes into account the sequence of the target site and/or its conformation in connection with the agent's action. Agents can be rationally selected or rationally designed by utilizing the peptide sequences that make up these sites.

The agents of the present invention can be, as examples, peptides, small molecules, vitamin derivatives, as well as carbohydrates. A skilled artisan can readily recognize that there is no limit as to the structural nature of the agents of the present invention.

The peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Another class of agents of the present invention are antibodies immunoreactive with critical positions of proteins of the invention. Antibody agents are obtained by immunization of suitable mammalian subjects with peptides, containing as antigenic regions, those portions of the protein intended to be targeted by the antibodies.

Atopy

Applicants have resolved the needs in the art by elucidating critical genes in the IL-9 pathway and compositions affecting that pathway which may be used in the diagnosis, prevention or treatment of atopic allergy including asthma and related disorders. Asthma encompasses inflammatory disorders of the airways with reversible airflow obstruction. Atopic allergy refers to atopy and related disorders including asthma, bronchial hyperresponsiveness, rhinitis, urticaria, allergic inflammatory disorders of the bowel and various forms of eczema. Atopy is a hypersensitivity to environmental allergens expressed as the elevation of serum total IgE or abnormal skin test responses to allergens as compared to controls. Bronchial hyperresponsiveness is a heightened bronchoconstrictor response to a variety of stimuli. Accordingly, the invention provides a purified and isolated DNA molecule comprising a nucleotide sequence encoding human or murine TH2AF1 or a fragment thereof the invention also includes degenerate sequences of the DNA as well as sequences that are substantially homologous. The source of TH2AF1 for the invention is human and murine. Alternatively, the DNA or fragment thereof, may be synthesized using methods known in the art. It is also possible to produce the agent by genetic engineering techniques, by constructing DNA using any accepted technique, cloning the DNA in an expression vehicle and transfecting the vehicle into a cell, which will express the agent. See, for example, the methods set forth in Sambrook et al., Molecular Cloning—A Laboratory Manual, Cold Spring Harbor Laboratory Press, 1985.

TH2AF1 Cloning

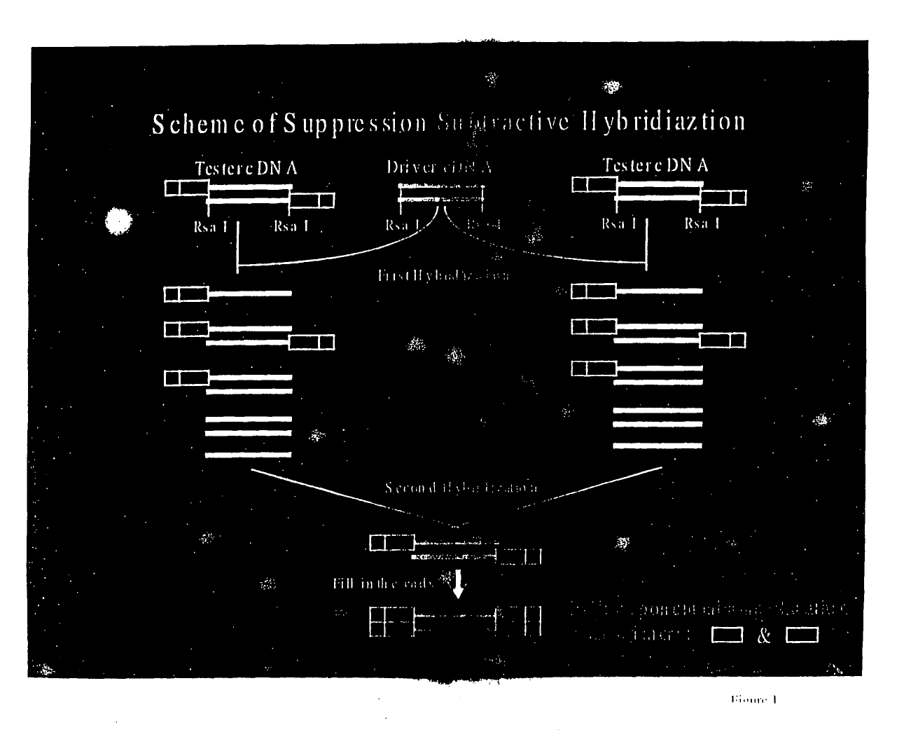

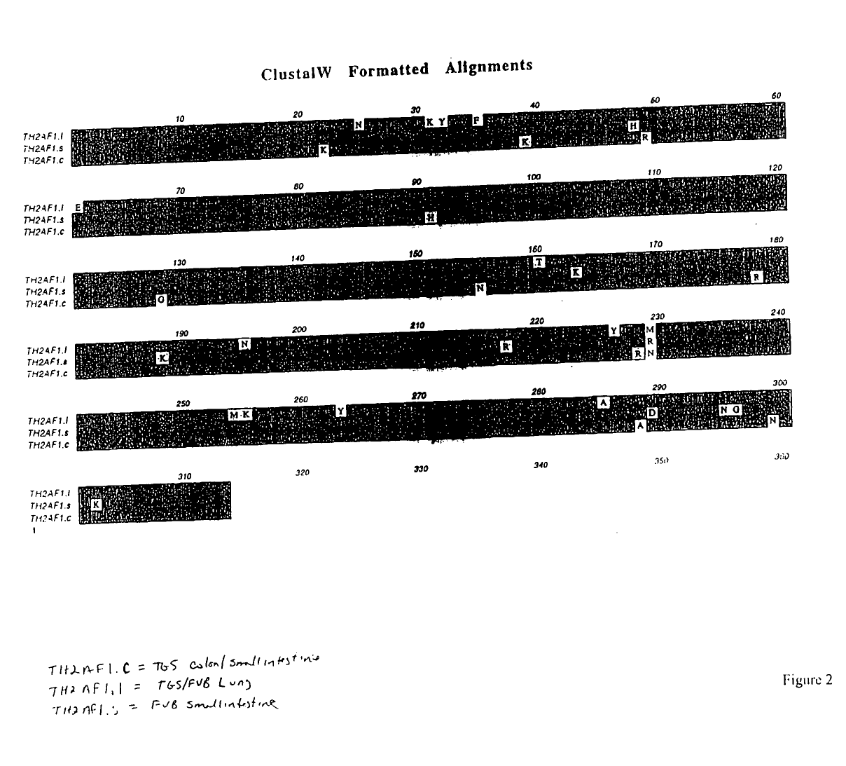

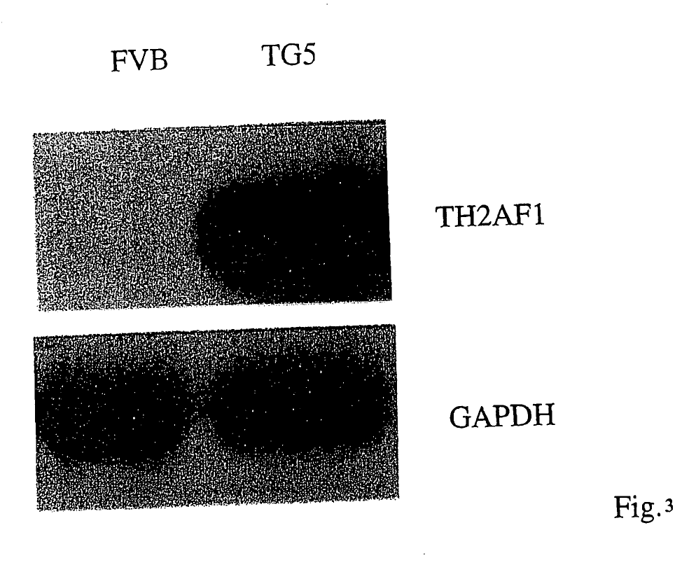

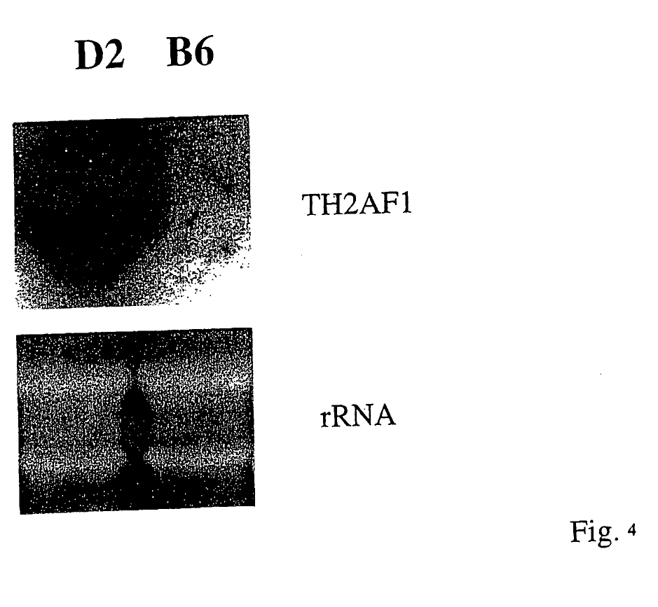

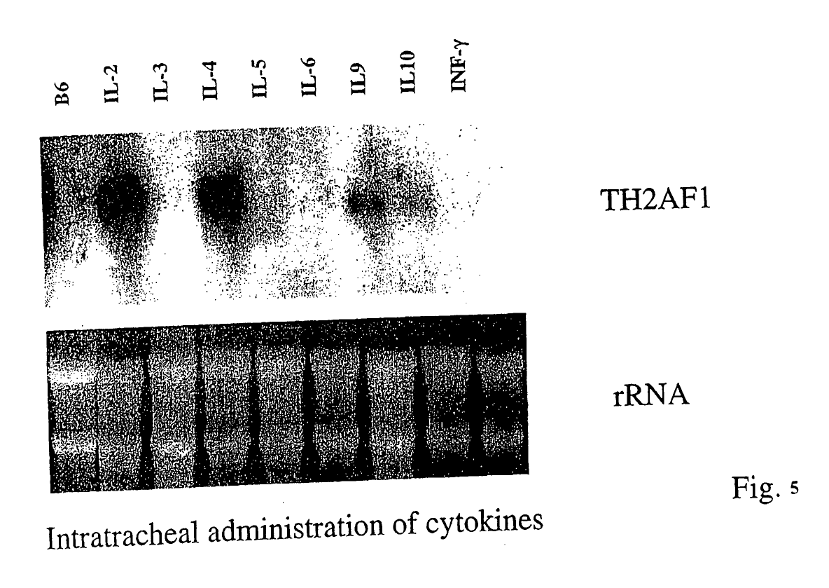

The murine TH2AF1 gene was identified by subtractive cDNA cloning experiments that were performed in order to identify genes specifically induced by IL-9. A schematic diagram of the subtractive cDNA cloning method is provided in FIG. 1. Applicants used RNA derived from lungs of transgenic mice over-expressing the murine IL-9 transgene (TG5) to isolate genes expressed in response to IL-9 as opposed to those which are not expressed in the parental strain (FVB). Library screening of mouse cDNA libraries and sequence analysis of recombinant clones derived from reverse transcriptase-polymerase chain reaction (RT-PCR) revealed a family of closely related TH2AF1 isoforms that are expressed preferentially in the gut of various mouse strains (FIG. 2 and SEQ ID NO: 2, 4, 6, 8, 10). Interestingly these proteins which are >98% identical are not found in the lung of naive animals except for the lung of IL-9 transgenic mice, demonstrating a tight correlation of the expression of the lung isoform and allergic and/or TH2 responses. FIG. 3 shows a Northern blot with RNA from a lung of a TG5 mouse (right lane) and a FVB mouse (left lane) demonstrating these findings. Expression of TH2AF1 was also observed in the lung of the DBA murine strain, which has been shown to express elevated baseline IL-9 levels in their lungs (FIG. 4). TH2AF1 expression was not observed in the lungs of the C57BL/6 strain where IL-9 expression is below the limits of detection (FIG. 4) (Nicolaides et al., (1997) Proc. Natl. Acad. Sci. USA 94, 13175-13180). The direct effect of IL-9 on inducing TH2AF1 expression was demonstrated when IL-9 was instilled into the trachea of the C57B6 mouse. The results of this experiment demonstrated that TH2AF1 was expressed in the lungs of the IL-9 instilled mice but not in naive or vehicle treated mice (FIG. 5), indicating that this gene is induced by IL-9. Moreover, TH2AF1 was found to be induced by other TH2-associated cytokines, such as IL-4 and IL-10, while TH1 associated cytokines such as interferon gamma did not induce the expression of this gene (FIG. 5).

The murine TH2AF1 gene displayed significant identity (62%) with a member of the Xenopus cortical granule Lectin family (Quill et al., (1996) Arch. Biochem. Biophys. 333, 326-332; Lee et al., (1997) Glycobiology. 7, 367-372). The full length murine cDNA was cloned using a cDNA RACE kit (FIG. 2) while cDNA was identified using BLAST analysis of the dbEST database at the National Center for Biotechnology Information to identify a partial sequence which was then fully cloned using 5′ and 3′ RACE (Clonetech).

Tissue expression of murine TH2AF1 was not detected in any tissue except in small intestine in naïve mice, while elevated expression of TH2AF1 was observed in lung, colon and small intestine in IL-9 transgenic mice, which over express this cytokine in all tissues (not shown). Interestingly, these tissues are comprised of epithelial cell types, suggesting that this gene may be restricted to IL-9 responsive epithelial cells. In situ analysis revealed that TH2AF1 expression was found in the epithelial cells of the lung in TG5 mice while no expression was observed in the naive congenic background strain (FVB) (not shown).

Further evidence defining the role of TH2AF1 in the pathogenesis of atopic allergy, bronchial hyperresponsiveness (BHR), asthma, and related disorders derives directly from the applicant's observation that IL-9 selectively induces TH2AF1. Thus, the pleiotropic role for IL-9, which is important to a number of antigen induced responses is dependent in part, on the up-regulation of TH2AF1 in cells critical to atopic allergy. When the functions of IL-9 are down-regulated by antibody pretreatment prior to aerosol challenge with antigen, animals can be completely protected from the antigen induced responses. These responses include: BHR, airway eosinophilia and elevated cell counts in bronchial lavage, histologic changes in lung associated with inflammation and elevated serum total IgE levels. Thus, treatment of such responses, which underlie the pathogenesis of atopic allergy and characterize allergic inflammation associated with this disorder, by down-regulating TH2AF1, is within the scope of this invention.

Applicants also teach the down-regulation of TH2AF1 by administering antagonists of TH2AF1. The skilled artisan will recognize that all molecules containing the requisite three-dimensional structural conformation critical for activation of, or soluble receptor and/or proteins binding to TH2AF1 are within the scope of this invention.

The demonstration of an IL-9 sequence associated with an asthma-like phenotype and one associated with the absence of an asthma-like phenotype, indicates that the inflammatory response to antigen in the lung is IL-9 dependent. Down-regulating TH2AF1, which is induced downstream in the IL-9 pathway, will therefore protect against this antigen-induced response. Furthermore, applicants also provide methods of diagnosing susceptibility to atopic allergy and related disorders and for treating these disorders based on the relationship between IL-9, its receptor and TH2AF1.

One diagnostic embodiment involves the recognition of variations in the DNA sequence of TH2AF1. One method involves the introduction of a nucleic acid molecule (also known as a probe) having a sequence complementary to TH2AF1 of the invention under sufficient hybridizing conditions, as would be understood by those in the art. In one embodiment, the sequence will bind specifically to one allele of TH2AF1 or a fragment thereof and in another embodiment will bind to both alleles. Another method of recognizing DNA sequence variation associated with these disorders is direct DNA sequence analysis by multiple methods well known in the art (Ott, (1991) Analysis of human genetic linkage, John Hopkins University Press). Another embodiment involves the detection of DNA sequence variation in the TH2AF1 gene associated with these disorders (Schwengel et al., (1993) Genomics 18, 212-215; Sheffield et al., (1993) Genomics 16, 325-332; Orita et al., (1989) Genomics 5, 874-879; Sarkar et al., (1992) Genomics 13, 441-443; Cotton, (1989) Biochem. J. 263, 1-10). These include the polymerase chain reaction, restriction fragment length polymorphism analysis and single stranded conformational analysis.

Specific assays may be based on monitoring the cellular functions of TH2AF1. Antagonists of the invention include those molecules that interact or bind to TH2AF1 and inactivate this protein. To identify other allosteric, inverse or weak antagonists of the invention, one may test for binding to TH2AF1. The present invention includes antagonists of TH2AF1 that block the function of this protein. Antagonists are agents that are themselves devoid of pharmacological activity but cause effects by preventing the action of an agonist. To identify an antagonist of the invention, one may test for competitive binding with natural TH2AF1 receptors or proteins that complex with TH2AF1 for activity. Assays of antagonistic binding and activity can be derived from monitoring TH2AF1 functions for down-regulation as described herein and in the cited literature. The binding of the antagonist may involve all known types of interactions including ionic forces, hydrogen bonding, hydrophobic interactions, van der Waals forces and covalent bonds. In many cases, bonds of multiple types are important in the interaction of an agonist or antagonist with a molecule like TH2AF1.

In a further embodiment, these agents may be analogues of TH2AF1 or soluble receptors. TH2AF1 analogues may be produced by point mutations in the isolated DNA sequence for the gene, nucleotide substitutions and/or deletions which can be created by methods that are all well described in the art (Simoncsits et al., (1994) Cytokine 6, 206-214). This invention also includes spliced variants of TH2AF1 and discloses isolated nucleic acid sequences of TH2AF1, which contain deletions of one or more of its exons. The term “spliced variants” as used herein denotes a purified and isolated DNA molecule encoding human TH2AF1 comprising at least one exon. There is no evidence of naturally expressed spliced mutants in the art. It mast be understood that these exons may contain various point mutations.

Structure-activity relationships may be used to modify the antagonists of the invention. For example, the techniques of X-ray crystallography and NMR may be used to make modifications of the invention. For example, one can create a three-dimensional structure of human TH2AF1 that can be used as a template for building structural models of deletion mutants using molecular graphics. These models can then be used to identify and construct a polypeptide or chemical structure for TH2AF1 which alters its normal function. In still another embodiment, these agents may also be used as dynamic probes for TH2AF1 structure and to develop TH2AF1 antagonists using cell lines or other suitable means of assaying TH2AF1 activity.

In addition, this invention also provides agents that prevent the synthesis or reduce the biologic stability of TH2AF1. Biologic stability is a measure of the time between the synthesis of the molecule and its degradation. For example, the stability of, a protein, peptide or peptide mimetic (Kauvar, (1996) Nature Biotech. 14, 709) therapeutic may be prolonged by using D-amino acids or shortened by altering its sequence to make it more susceptible to enzymatic degradation.

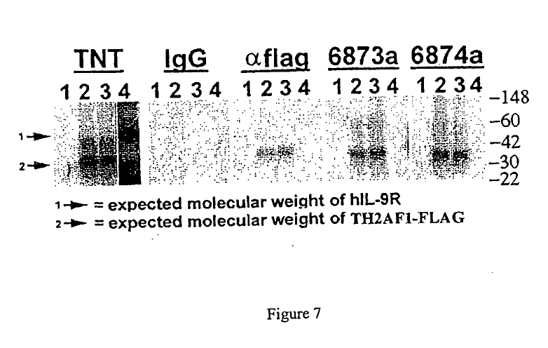

In another embodiment, antagonists of the invention are antibodies to TH2AF1 (see FIG. 7). The antibodies to TH2AF1 may be either monoclonal or polyclonal, made using standard techniques well known in the art (see Harlow & Lane, (1988) Antibodies—A Laboratory Manual, Cold Spring Harbor Laboratory Press). They can be used to block TH2AF1 activation by binding to extracellular regions of the protein required for ligand binding or activation. In one embodiment, the antibodies interact with TH2AF1, in another they interact with the receptor(s) for TH2AF1. The TH2AF1 used to elicit these antibodies can be the TH2AF1 protein or any of the TH2AF1 variants or fragments discussed above. Antibodies are also produced from peptide sequences of TH2AF1 using standard techniques in the art (see Protocols in Immunology, John Wiley & Sons, 1994).

In still another embodiment, the agents of the invention may be coupled to chemical moieties, including proteins that alter the functions or regulation of TH2AF1 for therapeutic benefit in atopic allergy and asthma (Kreitman et al., (1994) Biochemistry 33, 11637-11644). These proteins may include in combination other inhibitors of cytokines and growth factors including anti-IL-2, anti-IL-3, anti-IL-4, anti-IL-5, anti-IL-11, anti-IL-10 and anti-IL-13 that may offer additional therapeutic benefit in atopic allergy and asthma. In addition, the molecules of the invention may also be conjugated through phosphorylation to biotinylate, thioate, acetylate, iodinate using any of the cross-linking reagents well known in the art.

A further embodiment of the invention relates to antisense or gene therapy. It is now known in the art that altered DNA molecules can be tailored to provide a selected effect, when provided as antisense or gene therapy. The native DNA segment coding for TH2AF1 has two strands; a sense strand and an antisense strand held together by hydrogen bonds. The mRNA coding for the receptor has a nucleotide sequence identical to the sense strand, with the expected substitution of thymidine by uridine. Thus, based upon the knowledge of the receptor sequence, synthetic oligonucleotides can be synthesized. These oligonucleotides can bind to the DNA and RNA coding for TH2AF1. The active fragments of the invention, which are complementary to mRNA and the coding strand of DNA, are usually at least about fifteen nucleotides, more usually at least twenty nucleotides, preferably thirty nucleotides and more preferably may be fifty nucleotides or more. The binding strength between the sense and antisense strands is dependent upon the total hydrogen bonds. Therefore, based upon the total number of bases in the mRNA, the optimal length of the oligonucleotides sequence may be easily calculated by the skilled artisan.

The sequence may be complementary to any portion of the sequence of the mRNA. For example, it may be proximal to the 5′-terminus or capping site or downstream from the capping site, between the capping site and the initiation codon and may cover all or only a portion of the non-coding region or the coding region. The particular site(s) to which the antisense sequence binds will vary depending upon the degree of inhibition desired, the uniqueness of the sequence, the stability of the antisense sequence, etc.

In the practice of the invention, expression of TH2AF1 is down-regulated by administering an effective amount of antisense oligonucleotide sequences described above. The oligonucleotide agents of the invention bind to the mRNA coding for human TH2AF1 thereby inhibiting expression (translation) of these proteins. The isolated DNA sequences, containing various mutations such as point mutations, insertions, deletions are spliced mutations of TH2AF1 are useful in gene therapy as well.

In addition to the direct regulation of the TH2AF1 gene, this invention also encompasses methods of inhibition of intracellular signaling by TH2AF1. It is known in the art that highly exergonic phosphoryl-transfer reactions are catalyzed by various enzymes known as kinases. In other words, a kinase transfers phosphoryl groups between ATP and a metabolite. Included with the scope of this invention are specific inhibitors of protein kinase these kinases are useful in the down-regulation of TH2AF1 and are therefor useful in the treatment of atopic allergies and asthma. TH2AF1 is known to be glycosylated, therefore molecules that suppress the glycosylation of this protein may be useful for altering the function of TH2AF1.

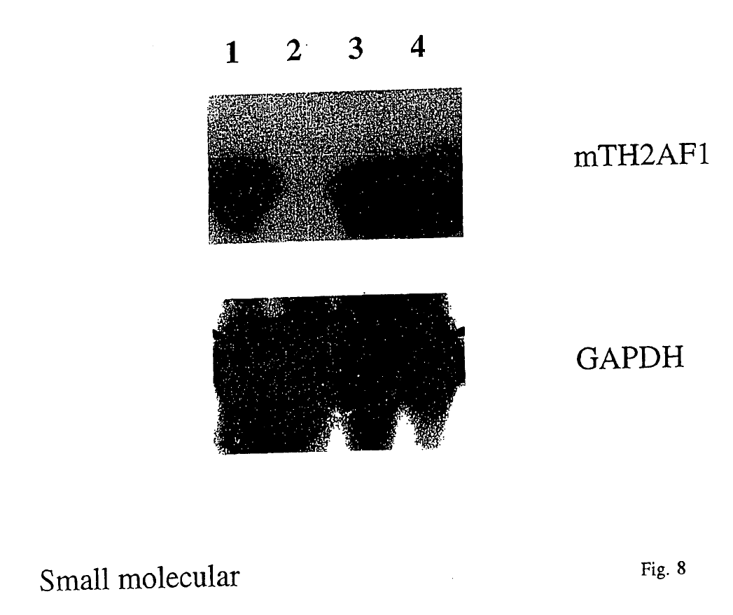

In still another aspect of the invention, surprisingly, aminosterol agents were found to be useful in the inhibition of TH2AF1 induction by IL-9 (FIG. 8). Aminosterol agents which are useful in this invention are described in U.S. patent application Ser. No. 08/290,826 and its related application Ser. Nos. 08/416,883 and 08/478,763 as well as U.S. patent application Ser. No. 08/483,059 and its related application Ser. Nos. 08/483,057, 08/479,455, 08/479,457, 08/475,572, 08/476,855, 8/474,799 and 08/487,443, which are specifically herein incorporated by reference. These related applications refer to the inhibitory activity of aminosterol agents in asthmatic-like responses in mouse models of asthma upon antigen exposure. Again these data reiterate the tight correlation of TH2AF1 in the asthmatic response and its expression is suppressed when the response is down regulated.

While a therapeutic potential for TH2AF1 down-regulation has been identified, applicants have also recognized a therapeutic potential for up-regulation of TH2AF1 as well. Autoimmune diseases have been found to be associated with a TH1 type of inflammation. These types of diseases, such as inflammatory bowel disease (IBD), have been previously shown to be treatable with the use of TH2-type proteins (Del Prete, (1998) Int. Rev. Immunol. 16, 427-455). The application of TH2AF1 as a pharmacologic agent for the treatment of this disease is suggested in part by its TH2-associated expression profile, and its induction in the cytokine IL-10, a TH2-type protein previously shown to have suppressive activity in IBD models (Opal et al., (1998) Clin. Infect. Dis. 27, 1497-507). Other autoimmune associated diseases such as diabetes, arthritis, ulcerative colitis, etc. are also potential diseases treatable by a recombinant TH2AF1 or fragment derived from this protein. A protein or fragment may consist of a modified structure that has enhanced pharmacological activity.

The invention also includes pharmaceutical compositions comprising the agents of the invention together with a pharmaceutically acceptable carrier. Pharmaceutically acceptable carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical carriers are described in Remington's Pharmaceutical Sciences, Mack Publishing (1995).

The agents used in the method of treatment of this invention may be a administered systemically or topically, depending on such considerations as the condition to be treated, need for site-specific treatment, quantity of drug to be administered and similar considerations.

Topical administration may be used. Any common topical formation, such as a solution, suspension, gel, ointment or salve and the like may be employed. Preparation of such topical formulations are well described in the art of pharmaceutical formulations as exemplified, for example, by Remington's Pharmaceutical Sciences. For topical application, these agents could also be administered as a powder or spray, particularly in aerosol form. The active ingredient may be administered in pharmaceutical compositions adapted for systemic administration. As is known, if a drug is to be administered systemically, it may be confected as a powder, pill, tablet or the like or as a syrup or elixir for oral administration. For intravenous, intraperitoneal or intra-lesional administration, the agent will be prepared as a solution or suspension capable of being administered by injection. In certain cases, it may be useful to formulate these agents in suppository form or as an extended release formulation for deposit under the skin or intramuscular injection. In a preferred embodiment, the agents of this invention may be administered by inhalation. For inhalation therapy the agent may be in a solution useful for administration by metered dose inhalers or in a form suit le for a dry powder inhaler.

An effective amount is that amount which will down-regulate TH2AF1. A given effective amount will vary from condition to condition and in certain instances may vary with the severity of the condition being treated and the patient's susceptibility to treatment. Accordingly, a given effective amount will be best determined at the time and place through routine experimentation. However, it is anticipated that in the treatment of atopic allergy and asthma-related disorders in accordance with the present invention, a formulation containing between 0.001 and five percent by weight, preferably about 0.01 to one percent, will usually constitute a therapeutically effective amount. When administered systemically, an amount between 0.01 and 100 milligrams per kilogram body weight per day, but preferably about 0.1 to 10 milligrams per kilogram, will effect a therapeutic result in most instances.

The practice of the present invention will employ the conventional terms and techniques of molecular biology, pharmacology, immunology and biochemistry that are within the ordinary skill of those in the art. For example, see Sambrook et al., (1985) Molecular Cloning—A Laboratory Manual, Cold Spring Harbor Laboratory Press).

In still another aspect of the invention, TH2AF1 gene or protein expression may serve as diagnostic markers for the detection of airway inflammatory diseases such as asthma, and a markers for inflammatory diseases of the gut, where a constitutive expression of TH2AF1 is found in both human and murine tissues.

Other embodiments of the invention will be apparent to those skilled in the art from consideration of the specification and practice of the invention disclosed. It is intended that the specifications and examples be considered exemplary only with a true scope of the invention being indicated by the claims. Having provided this background information applicants now describe preferred aspects of the invention TH2AF1.

### Example 1

### cDNA Difference Analysis of IL-9 Expressed Genes

Lungs extracted from transgenic IL-9 mice (TG5) were used to isolate IL-9 induced genes. TG5 is a FVB mouse overexpressing the IL-9 gene as previously described (Renauld et al., (1994) Oncogene 9, 1327-1332). This strain has been shown to overexpress IL-9 in most tissues of the mouse. In order to identify specific IL-9 induced genes, suppressive PCR cDNA difference analysis was performed on mRNA from lungs of TG5 mice and parental FVB mice using a commercially available PCR-select cDNA subtraction kit (Clonetech).

cDNA synthesis. Total RNA was prepared from lungs of FVB and TG5 mice using Trizol as described by the manufacturer (Gibco-BRL). Lungs were removed from euthanized mice and frozen in liquid nitrogen. Frozen lungs were then placed in Trizol and pulverized using a tissue grinder. Polyadenylated RNA was purified from total RNA with oligo(dT) cellulose columns (Pharmacia). Double stranded cDNA was prepared using Superscript II reverse transcriptase and an oligo(dT) primer as suggested by the manufacturer (Clonetech). cDNA was then prepared by phenol-chloroform extraction and ethanol precipitation. Products were resuspended in nuclease-free water and analyzed on agarose gels to determine quality of products as described below.

Differential cDNA analysis of TG5 and FVB lungs was carried out following the manufacturer's protocol (Clonetech) as depicted in FIG. 1. The results of the subtraction between the cDNA of these lungs resulted in the generation of 1200 recombinant clones. Analysis of these clones revealed multiples of several species, each accounting for 2-5% of the library. The most prominent transcript in the library was the IL-9 cDNA which served as a control for the efficiency of subtraction since it was a subtraction between an IL-9 constitutively expressing mouse (TG5) and its parental control.

### Example 2

### Identification of the Murine TH2A.F1 cDNA in the Lung of IL9 Transgenic Mice

Of 836 clones which yielded valid sequence, a novel cDNA has been recovered 17 times. A nucleotide BLAST (Altschul et al., (1990) J. Mol. Biol. 215, 403-410) database search of GenBank with this 321 base fragment revealed that it was most similar to Xenopus cortical granule lectin protein, and it was similar to several mouse EST clones. TH2AF1 primers derived from this 321 base cDNA and from the mouse EST clones were used to do RACE from a lung cDNA RACE kit (Marathon-Ready cDNA, Balb/c male) according to the manufacturer's recommendation (Clonetech). For 5′ RACE, antisense primers mLectin7 (SEQ ID NO: 13), 5′-GGGTTCTTGTAGTCATCACTTGTGGCAG-3′, mLectin9 (SEQ ID NO: 14), 5′-TGCAGACCCAAAGGTGTTGTAGTTGGC-3′ were used; for 3′ RACE, antisense primers mLectin10 (SEQ ID NO: 15), 5′-CTGCCACAAGTGATGACTACAAGAACCC-3′, mLectin8 (SEQ ID NO: 16), 5′-CGTGCAGTGTGGAGACTTTGCTGCA-3′ were used. The PCR fragment were cloned into TA cloning vector and sequenced using primers directed to the plasmid vector as well as internal sequences identified from the partially subtracted DNA fragment and sequence derived from the mouse EST clones. Sense primer mLectin20 (SEQ ID NO: 17), 5′-GAAAGGTTCCTGTCATTACTCAGC-3′, antisense primer mLectin23 (SEQ ID NO: 18), 5′-CTGCTTTATTGCTCATTAGCATTC-3′ generated from these 5′ and 3′ RACE sequence were used to generated PCR product from lung of FVB and TG5 (SEQ ID NO: 1) and small intestine of FVB (SEQ ID NO: 3), TG5 (SEQ ID NO: 5), DBA2 (SEQ ID NO: 7) and C57BL/6 (SEQ ID NO: 9) mouse. PCR products were cloned and sequenced. Clones were then aligned and contiged to generate the cDNA sequence containing the full ORF. Apparently, besides from strain variation, it is very clear that each mouse strain has at lease three kinds of highly homologous TH2AF1. This is probably due to an ancient chromosomal duplication event. A protein alignment of the mouse TH2AF1 isoforms is provided in FIG. 2. These isoforms have a >98% identity at the amino acid level and suggest a conserved function for these proteins. The primary sequence of murine TH2AF1 was used to perform an expressed sequence tag (EST) database search for human sequences and several undescribed human ESTs were found to be similar to the novel cDNA. A 5′ human TH2AF1 sequence was generated using a human small intestine RACE kit (Marathon-Ready cDNA, Clonetech) according to the manufacturer's recommendation (SEQ ID NO: 11). Antisense primers hLectin7 (SEQ ID NO: 19), 5′-AGGGTTCTTGTAGTCATCGCTCGTGG-3′, hLectin9 (SEQ ID NO: 20), 5′-AGATCCAAAGGTGTTGTAGTTGGCCC-3′ were used for 5′ RACE. Antisense primer hLectin2 (SEQ ID NO: 21), 5′-GCTCTAGATCTCATGGTTGGGAGGAGGG-3′ was derived from a human EST clone. hLectin2 and sense primer hLectin16 (SEQ ID NO: 22), 5′-GAAAGCTGCACTCTGTTGAGC-3′ were used to generated PCR product from this human small intestine RACE kit.

PCR products were cloned and sequenced. Clones were then aligned and contiged to generate the cDNA sequence containing the full ORF of human TH2AF1 (SEQ ID NO: 11 & 12). Primers hLectin2 (SEQ ID NO: 21) and sense primer hLectin18 (SEQ ID NO: 23), 5′-GCAGCTGAGACTCAGACAAG-3′ were also used to generate PCR product from RT-cDNA sample of A549 treated with IL-4 (A549 is a human lung epithelium cell line). The human TH2AF1 sequence derived from A549 is exactly the same as that from small intestine (SEQ ID NO: 11). The human TH2AF1 homolog was found to be highly conserved with the mouse (81% identity) and Xenopus (60% identity) proteins. Motif analysis of the encoded protein demonstrated several features such as secretion signal peptide (codons 1-18) and glycosylation sites.

### Example 3

### TH2AF1 is Induced In Vivo by IL-9 in Murine Cells

To confirm that TH2AF1 is induced by IL-9 in the lung, RNA from the lungs of TG5 and FVB mice were isolated as described in Example 1. cDNA was generated using random hexamers (Pharmacia) and Superscript II (Gibco-BRL) as suggested by the manufacturer. Message was analyzed by PCR as described (Nicolaides et al., (1995) Genomics 30, 195-206) and via Northern blot. Primers used to generate murine TH2F1 message were; sense mLectin20 (SEQ ID NO: 17) (nucleotides) and antisense mLectin23 (SEQ ID NO: 18) which produce a gene product of 1,104 base. GAPDH was assayed as an internal control to measure for cDNA integrity using primers previously described (Nicolaides et al., (1991) Genomics 30, 195-206). Amplification conditions used were 94° C. for 30 seconds, 58° C. for 1.5 minutes and 72° C. for 1.5 minutes for 35 cycles. Via Northern blot analysis, total RNA derived from TG5 or FVB lungs was electrophoresed on 1.5% formaldehyde gels, transferred to nylon membranes and probed with a DNA fragment comprising the murine TH2AF1 cDNA.

The results of the expression studies demonstrated that TH2AF1 is specifically expressed in the lung of the IL-9 transgenic (TG5) mouse but low or no expression is observed in the parental strain (FVB) (FIG. 3). GADPH (lower panel) was used as an internal control to assess for RNA loading. This data demonstrated a direct effect of IL-9 on TH2AF1 expression in the lung, where IL-9 responsive cells contained within the lung express TH21 AF1.

### Example 4

### TH2AF1 Expression can be Induced in the Murine Lung by IL-9

TH2AF1 gene expression was assessed in vivo using the C57BL/6 mouse which does not express detectable levels of IL-9 and the DBA mouse which expresses robust levels of IL-9 (Nicoiaides et al., (1997) Proc. Natl. Acad. Sci. USA 94, 13175-13180. RT-PCR and Northern blot analysis of TH2AF1 from these lungs demonstrated that TH2AF1 was expressed in the lung of mice which naturally express high levels of IL-9 (DBA) but not in those with low levels of IL-9 (C57BL/6) (FIG. 4). Ribosomal RNA (rRNA, lower panel) was stained using ethidium bromide (lower panel) to control for RNA loading.

To confirm that the expression of IL-9 was critically related to the expression of TH2AF1 and to control for genetic background specifically, recombinant murine IL-9 was introduced into the lung of murine strain C57BL/6. Recombinant IL-9 was instilled into the trachea of anesthetized mice by addition of 50 μl of a 0.1 mg/ml IL-9 solution or vehicle alone (0.1% bovine serum albumin) daily for ten days. After ten days, the mice were euthanized and lungs extracted for either RNA expression analysis using Trizol as described by the manufacturer (Gibco-BRL) or Western blot analysis to determine levels of IL-9 instilled. The Western blot analysis for IL-9 demonstrated that direct addition of IL-9 to the lung result d in an increase of overall amount of IL-9 in the lung while none was observed in the mouse instilled with vehicle alone (Shimbara et al., (2000) J. Allergy Clin. Immunol. 105, 108-115).

Expression of TH2AF1 RNA was measured as described in Example 3. Steady state mRNA analysis for TH2AF1 expression indicated that expression increased when recombinant IL-9 was administered to the lungs of the C57BL/6 mice, while no expression was observed in the lungs of mice treated with vehicle only (FIG. 5). This data demonstrates a direct role of IL-9 on inducing TH2AF1 expression in the lung. The addition of other recombinant cytokines suggested a pattern of gene induction, where TH2 associated cytokines IL-4, IL-9, and IL-10 all induced the expression of TH2F1 when instilled into the airway of C57BL/6 mice but not TH1 associated cytokines such as interferon gamma (INF-γ). Ribosomal RNAs (rRNA, lower panel) were stained using ethidium bromide (lower panel) to control for RNA loading.

### Example 5

### Tissue Distribution of TH2AF1 in Mice

To address the possibility that TH2AF1 expression occurs only in the presence of IL-9 expression, various organs were extracted from TG5 mice and analyzed for RNA expression via Northern blot. The parental strain FVB mice were used as a control because they express low levels of IL-9 in the lung when compared to TG5 mice. Tissue blots for TG5, FVB murine organs were prepared by extracting organs followed by freezing in liquid nitrogen. Total RNA was extracted from each of these organs using Trizol as described by the manufacturer (Gibco-BRL). RNA was gel electrophoresed and analyzed as described in Example 4. Lanes were standardized by probing with GAPDH as an internal control.

Tissue blots were probed using a DNA fragment comprising the TH2AF1 cDNA. Robust steady state TH2AF1 expression was found only in the small intestine in naive mice (not shown). Analysis of TH2AF1 expression in organs derived from the IL-9 transgenic ice revealed high level expression in the lung, colon as well as in the small intestine (not shown). This data demonstrated that TH2AF1 is expressed in several tissues in mice overexpressing IL-9 but only constitutively in small intestine in those with low IL-9 levels. This data suggests that TH2 AF1 may play a role in the physiology of these organs in response to IL-9. The induction of TH2AF1 in the gut might indicate a role of TH2AF1 in the inflammatory bowel disease. Expression of the human homolog was also observed in the gut of man. In situ gene expression using lung tissue from IL-9 transgenic and congenic background strain mice revealed that the expression of TH2AF1 is from the epithelia of the airway suggesting that epithelial cells are the normal producers of this factor in response to a subset of TH2 cytokines (not shown).

### Example 6

### Antigen Induced TH2AF1 Expression in Mice Exhibiting an Asthmatic-like Phenotype

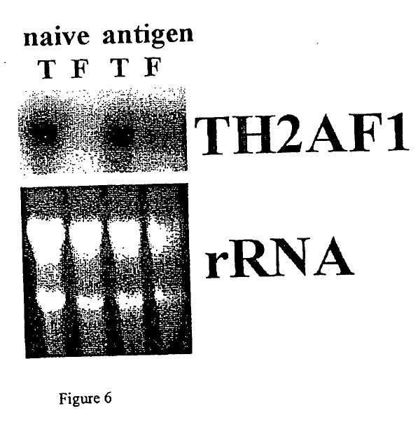

Gene expression profiling shows that TH2AF1 is expressed in the lung of naïve IL-9 transgenic (TG5)-mice but not the congenic control strain (FVB) (FIG. 3). Moreover, its expression was observed in the lung of the hyperresponsive DBA/2 mouse (FIG. 4). To determine if TH2AF1 expression is associated with the asthmatic-like lung of antigen exposed mice, steady-state mRNA levels were assayed in the lung of naive and antigen exposed FVB and TG5 mice which exhibit increased BHR, lung eosinophilia and elevated total serum IgE levels upon antigen exposure (McLane et al., (1998) Am. J. Respir. Cell Mol. Biol., 19, 713-720). These mice exhibit increased eosinophils in the airway after antigen exposed IL-9 transgenic mice (T) (3.3×10⁵) as compared to the FVB congenic background strain mice (F) (3.3×10⁴) and increased airway hyperresponsiveness in both animals (McLane et al., (1998) Am. J. Respir. Cell Mol. Biol., 19, 713-720). FIG. 6 shows the steady state expression level of TH2AF1 is also increased in the FVB control mouse (F lane) upon antigen exposure, while expression in the enhanced TG5 mouse (T lane) remains robust and is associated with the enhanced asthmatic-like responses. This data demonstrates a tight expression pattern of TH2AF1 with the airway inflammation and suggests a role for this gene in regulating the disease process.

### Example 7

### Generation of Antibodies to TH2AF1

Rabbits were vaccinated with a peptide comprising C-terminal residues 302 to 313 of TH2AF1 which are identical between mouse and human proteins. Briefly, animals were prebled for 3-5 milliliters of serum per rabbit, then injected with antigen on day 1, 7, 14, 28, 56, and 84 in Freund's adjuvant. Serum samples were taken on days 42, 70 and 80 and analyzed for antiTH2AF1 titers against the peptide used for immunization and the whole molecule by plate ELISA. Briefly, ELISAs were carried out using 96 well microtiter plates coated with a 1 μg/ml solution containing, peptide, recombinant TH2AF1 or a nonspecific antigen in PBS (pH 7.4) in triplicate wells. Coating reagent was removed and wells were washed three times using PBS (pH 7.4) plus 0.01% Tween-20, and blocked in 5% BSA in PBS (BSA-PBS) for two hours at room temperature. Plates were then washed and incubated with serial dilutions (0.1, 0.01, 0.001) of rabbit pre-immune or active antisera in BSA-PBS for one hour. Plates were the n washed three times and incubated with anti-rabbit horseradish conjugated antibody diluted 1:3000 in PBS-BSA for thirty minutes. Finally, plates were washed and incubated with TMB peroxidase substrate (BioRAD) for thirty minutes, and analyzed at 450 nm on a Dynatech plate reader. Two antisera were found to have good titers against the peptide antigens (6873 and 6874) and were further evaluated using in vitro translated murine and human TH2AFT1 containing a FLAG epitope tag at the C-terminus as positive control for the ability of this antiserum to detect TH2AF1 by immunoprecipitation (IP) in vitro. The murine and human TH2AF1-FLAG proteins were generated from transcripts obtained by PCR amplification of human and mouse TH2AF1 cDNA using a sense primer, which contains a T7 promoter and Kozak consensus sequence, for initiation of translation and an antisense primer that contains the FLAG epitope (underlined, see below) fused to the last amino acid of the mature peptide followed by a termination codon. The sense primer is targeted against codons 19-24, where codon 19 is the first residue of the mature TH2AF1. The C-terminal FLAG fusion epitope serves as a positive control where the monoclonal anti-FLAG (IBI) is also used for detection.

The primers used were,

murine sense:

(SEQ ID NO:24)

5′-ggatcctaatacgactcactatagggagaccaccatggcagctgaa
gactg-3′;
Murine antisense:

(SEQ ID NO:25)

5′-ggtccttatcacffgtcqtcgtcgtcctcgcgatagaatag-3′;
Human sense:

(SEQ ID NO:26)

5′-ggatcctaatacgactcactatagggagaccaccatgagtacagatg
aggctaatacttac-3′;
Human antisense:

(SEQ ID NO:27)

5′-ggatccttatcacggtcatcgtcgtccttagtcgatagaatagaaga
cagc-3′.

PCR amplifications were carried out at 94° C. for 30 seconds, 54° C. for one minute, 72° C. for one minute for 30 cycles in a Hybaid 96-well Touchdown thermocycler using Taq-Pfu polymerase, which has an increased fidelity in DNA replication. PCR products of approximately one kilobase in length, were then phenol chloroform extracted, ethanol precipitated and resuspended at a concentration of 1 μg/μl in RNAse & DNAse-free water as templates for in vitro transcription coupled translation (TNT) reactions. For TNT reactions, 1 μg of template was added to 50 μl of reticulolysate mix (Promega) containing radiolabeled ³⁵S-methionine. Reactions were carried out for one hour at 30° C. Human IL-9 receptor cDNA was PCR amplified and in vitro translated for use as a negative control as well as reticulolysates not programmed with template. After incubation 5 μl of each reaction was run on a 4-20% Tris-Glycine SDS-PAGE gel (Novex), fixed with 5% methanol and 5% acetic acid, dried, and autoradiographed. FIG. 7 (TNT panel) shows a typical result from in vitro translation reactions. Equal amounts of translated products were then used for IP using antisera from rabbit prebleeds (IgG panel), TH2AF-1 antisera 6873 and 687 and anti-FLAG. Samples are diluted to 300 μl in EBC buffer (0.1 MnCl, 50 mM Tris-HCl, pH 7.5, 0.5% NP40) and 10 μl of antiserum or 1 μg of anti-FLAG were then added and incubated on ice for one hour. 40 μl of Protein A-Sepharose (Sigma) was added to each sample, and samples were tumbled for 12 hours at 4° C. After incubations were complete, samples were microfuge at 14,000 RPM and washed five times with EBC. After the last wash, pellets were resuspended in 2×SDS buffer (60 mM Tris, pH 6.85, 2% SDS, 10% glycerol, 0.1 M 2-mercaptoethanol, 0.001% bromophenol blue), boiled for five minutes and run on a 4-20% Tris-Glycine SDS-PAGE gel, fixed, dried and autoradiographed. FIG. 7 shows a typical experiment where TH2AF1 antisera (#6873 and #6874) were able to specifically recognize human and mouse TH2AF-1 but not human IL-9 receptor.

### Example 8

### Blocking of TH2AF1 Induction by Aminosterols in Murine Lung

Lungs from the DBA bronchial hyperresponsive mouse are treated with aminosterol agents to test for their ability to block expression of TH2AF1. This group of aminosterols was identified from the liver of the dogfish shark as a class of molecules that appear to be antiproliferative. An example of these agents are referred to in related U.S. Pat. No. 5,637,691 and its related U.S. Pat. Nos. 5,733,899 and 5,721,226 as well as in U.S. Pat. No. 5,840,740 and its related U.S. Pat. Nos. 5,795,885, 5,994,336, 5,763,430, 5,840,936, 5,874,597, 5,792,635 and 5,847,172. Members of this series of aminosterols were assayed for their ability to inhibit TH2AF1 expression and TH2 activity from the DBA mouse as described below.

DBA/2 mice were injected daily intraperitoneally with various aminosterols at 10 mg/kg for fifteen days. At day fifteen, mice were phenotyped (see Example 9), euthanized and lungs extracted as described in Example 1. RNA was isolated and processed for Northern blot analysis using a TH2AF1 cDNA probe. Steady-state TH2AF1 RNA levels indicate the extent of inhibition by aminosterol treatment when compared to control. The ability of specific aminosterols, such as FIG. 8 indicates that 1432 to block the expression of TH2AF1 in vivo. FIG. 8 indicates that 1432 (lane 2) can down-regulate the expression of TH2AF1 in the lung of treated mice in contrast to mice treated with the corticosteroid dexamethasone (lane 3) which has minimal effects on suppressing asthmatic-like responses to antigen in this model. As shown in previous experiments (FIG. 4), DBA/2 mice which are naturally airway hyperresponsive (Nicolaides et al., (1997) Proc. Natl. Acad. Sci. USA 94, 13175-13180) exhibit a baseline TH2AF1 expression (lane 1) which is enhanced upon antigen exposure (lane 4). This data shows a tight correlation of TH2AF1 expression and the asthmatic-like lung. GADPH was measured (lower panel) to control for RNA loading.

### Example 9

### Role of TH2AF1 in Murine Models of Asthma

### Airway Response of Unsensitized Mice

Certified virus-free male and female mice of the following strains, DBA, C57136 and B6D2F1 are purchased from the National Cancer Institute or Jackson Laboratories (Bar Harbor, Me.). IL-9 transgenic mice (TG5) and their parent strain (FVB), are obtained from the Ludwig Institute (Brussels, Belgium). Animals are housed in high-efficiency particulate filtered air laminar flow hoods in a virus and antigen free facility and allowed free access to food and water for three to seven days prior to experimental manipulation. The animal facilities are maintained at 22° C. and the light:dark cycle is automatically controlled (10:14 hour light:dark).

To determine the bronchoconstrictor response, respiratory system pressure is measured at the trachea and recorded before and during exposure to the drug. Mice are anesthetized and instrumented as previously described. (Levitt et al., (1988) FASEB J. 2, 2605-2608; Levitt et al., (1991) Pharmacogenetics 1, 94-97; Kleeberger et al., (1990) Am. J. Physiol. 258, 313-320; Levitt et al., (1995) Am. J. Respir. Crit. Care. Med. 151, 1537-1542; Ewart et al., (1995) J. Appl. Phys. 79, 560-566). Airway responsiveness is measured to one or more of the following: 5-hydroxytryptamine, acetylcholine, atracurium or a substance-P analog. A simple and repeatable measure of the change in peak inspiratory pressure following bronchoconstrictor challenge is used which has been termed the airway Pressure Time Index (APTI) (Levitt et al., (1988) FASEB J. 2, 2605-2608; Levitt et al., (1989) J. Appl. Physiol. 67, 1125-1132). The APTI is assessed by the change in peak respiratory pressure integrated from the time of injection until the peak pressure returns to baseline or plateau. The APTI is comparable to airway resistance, however the APTI includes an additional component related to the recovery from bronchoconstriction.

Prior to sacrifice, whole blood is collected for serum IgE measurements by needle puncture of the inferior vena cava in anesthetized animals. Samples are centrifuged to separate cells and serum is collected and used to measure total IgE levels. Samples not measured immediately are frozen at −20° C.

All IgE serum samples are measured using an ELISA antibody-sandwich assay. Microtiter plates are coated, 50 μl per well, with rat anti-murine IgE antibody (Southern Biotechnology) at a concentration of 2.5 μg/ml in a coating buffer of sodium carbonate-sodium bicarbonate with sodium azide. Plates are covered with plastic wrap and incubated at 40° C. for sixteen hours. The plates are washed three times with a wash buffer of 0.05% Tween-20 in phosphate-buffered saline, incubating for five minutes for each wash. Blocking of nonspecific binding sites is accomplished by adding 200 μl per well 5% bovine serum albumin in phosphate-buffered saline, covering with plastic wrap and incubating for two hours at 37° C. After washing three times with wash buffer, duplicate 50 μl test samples are added to each well. Test samples are assayed after being diluted 1:10, 1:50 and 1:100 with 5% bovine serum albumin in wash buffer. In addition to the test samples, a set of IgE standards (PharMingen) at concentrations from 0.8 ng/ml to 200 ng/ml in 5% bovine serum albumin in wash buffer, are assayed to generate a standard curve. A blank of no sample or standard is used to zero the plate reader (background). After adding samples and standards, the plate is covered with plastic wrap and incubated for two hours at room temperature. After washing three times with wash buffer, 50 μl of secondary antibody rat anti-murine IgE-horseradish peroxidase conjugate is added at a concentration of 250 ng/ml in 5% bovine serum albumin in wash buffer. The plate is covered with plastic wrap and incubated two hours at room temperature. After washing three times with wash buffer, 100 μl of the substrate 0.5 ng/ml o-phenylenediamine in 0.1 M citrate buffer is added to every well. After ten minutes the reaction is stopped with 50 μl of 12.5% sulfuric acid and absorbance is measured at 490 nm on a MR5000 plate reader (Dynatech). A standard curve is constructed from the standard IgE concentrations with antigen concentration on the x-axis (log scale) and absorbance on the y-axis (linear scale). The concentration of IgE in the samples is interpolated from the standard curve.

Bronchioalveolar lavage and cellular analysis are preformed as previously described (Kleeberger et al., (1990) Am. J. Physiol. 258, 313-320). Lung histology is carried out after the lungs are extracted. Since prior instrumentation may introduce artifact, separate animals are used for these studies. Thus, a small group of animals is treated in parallel exactly the same as the cohort undergoing various pretreatments except these animals are not used for other tests aside from bronchial responsiveness testing. After bronchial responsiveness testing, the lungs are removed and submersed in liquid nitrogen. Cryosectioning and histologic examination is carried out in a manner obvious to those skilled in the art.

Polyclonal antibodies which block the murine TR2AF1 pathway are used therapeutically to down-regulate the functions of, and assess the importance of this pathway to bronchial responsiveness, serum IgE and bronchioalveolar lavage in sensitized and unsensitized mice. After antibody pretreatment, baseline bronchial hyperresponsiveness, bronchioalveolar lavage and serum IgE levels relative to Ig matched controls are determined.

### Example 10

### Role of TH2AF1 in Murine Models of Asthma

### Airway Response of Sensitized Mice

Animals and handling are essentially as described in Example 8. Sensitization by nasal aspiration of Aspergillus fumigatus antigen (1:100 dilution) is carried out to assess the effect on bronchial hyperresponsiveness, bronchioalveolar lavage and serum IgE. Mice are challenged with Aspergillus or saline intranasally (Monday, Wednesday and Friday for three weeks) and phenotyped twenty-four hours after the last dose. The effect of pretreatment with TH2AF1 antibodies is used to assess the effect of down-regulating TH2AF1 in mice.

### Example 11

### Blocking of TH2AF1 Signaling In Vivo by Anti-Murine IL-9 Antibody

Animals and handling were essentially as described in Example 8. Sensitization by nasal aspiration of Aspergillus fumigatus antigen (1:100 dilution) was carried out to assess the effect on bronchial hyperresponsiveness, bronchioalveolar lavage and serum IgE. Mice were challenged with Aspergillus or saline intranasally (Monday, Wednesday and Friday for three weeks) and phenotyped twenty-four hours after the last dose. The effect of pretreatment with IL-9 antibodies was used to assess the effect of down-regulating TH2AF1 in mice. These studies showed that pretreatment with IL-9 antibody can down-regulate TH2AF1 expression level in mouse lung (not shown).

### Example 12

### TH2AF1 is a Secreted Factor

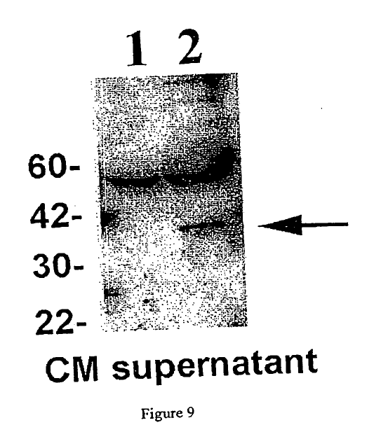

To test if TH2AF1 is a secreted protein as is the case for the Xenopus and Roach homologs (Nishihara et al., (1986) Biochem. 25, 6013-6020; and Licastro, et al., (1991) Int. J. Biochem. 23, 101-105), the entire coding region of the full-length murine TH2AF1 cDNA was cloned into the pcDNA expression vector that contains the CMV promoter followed by a polylinker cloning site and a polyadenylation signal. This vector also contains a neomycin resistance gene, which allows for the selection of stable transfected cells. Human embryonic kidney 293 (HEK293) cells were transfected using lipofectamine as suggested by the manufacturer's protocol (Gibco-BRL). Cells transfected with nothing (mock), empty vector and TH2AF1 expression vector were selected for two weeks in medium containing G418. G418 resistant cells grew in cultures transfected with empty and TH2AF1 expression vectors, but not in mock transfected cells. Cultures were then tested for TH2AF1 gene expression using the antisera described in Example 7. Analysis of conditioned medium (CM) from cells transfected with the TH2AF1 expression vector found protein in the CM from these cells in contrast to cells transfected with empty vector (FIG. 9). The figure shows a Western blot of CM derived from 293 cells stably expressing TH2AF1 (lane 2) or empty vector (lane 1). The arrow indicates a band of expected molecular weight.

### Example 13

### TH2AF1 Activates Human Lymphocytes

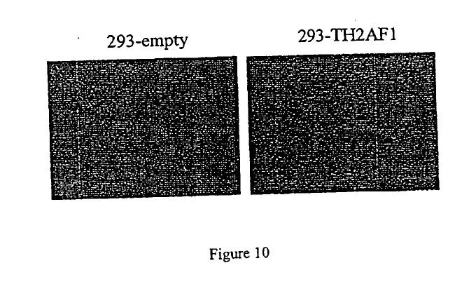

A TH2AF1 homolog from Roach oocytes can stimulate the mitogenic activity of human lymphocytes (Licastro et al., (1991) Int. J. Biochem. 23, 101-105; Komiya et al., (1998) Biochem. Biophys. Res. Comm. 251, 759-762). CM from cells expressing empty vector or TH2AF1 that are described in Example 12 were used to determine if TH2AF1 could activate human PBMC as is the case for the Xenopus and Roach homologs (Nishihara et al., Biochemistry 25, 6013-6016; Licastro et al., (1991) Int. J. Biochem. 23, 101-105). Peripheral blood mononuclear cells (PBMC) obtained from a healthy volunteer by venipuncture were used. Peripheral blood was diluted 1:4 in phosphate buffered saline solution and mononuclear cells were isolated by centrifugation over a ficoll-hypaque gradient as described (Grasso et al., (1998) J. Biol. Chem., 273, 24016-24024). Cells were then plated at 1×10⁵ cell/well in twenty-four well plates in growth medium (RPMI-1640 plus 10% heat inactivated fetal bovine serum) supplemented with or without 10% (final volume) of CM from empty vector or TH2AF1 transfected cells (see Example 12) and cultures were grown at 37° C. in 5% sera for twenty-four hours. FIG. 10 shows that CM from 293-TH2AF1 cells resulted in cellular activation of PBMC cultures as indicated by cellular aggregates (indicated arrows) in contrast to cultures grown in the presence of empty vector or medium alone. These data suggest that TH2AF1 is a factor that is involved in activating lymphocytes that in vivo are associated with humoral type responses (see Example 6).

### Example 14

### Recombinant Production of TH2AF1 and Biological Activity on Human Lymphocytes

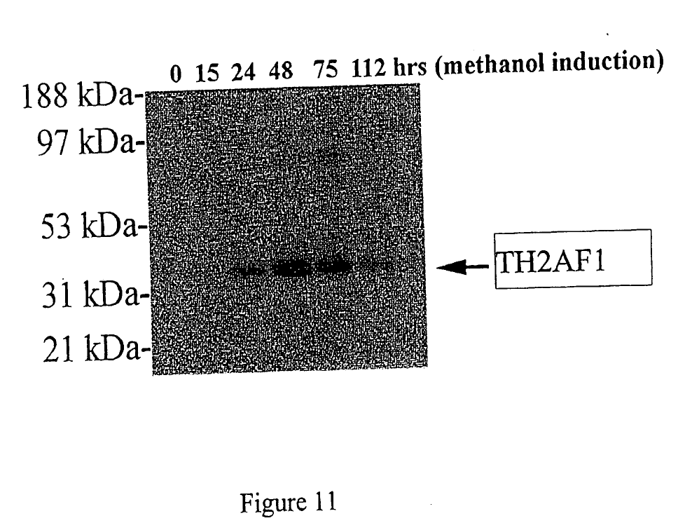

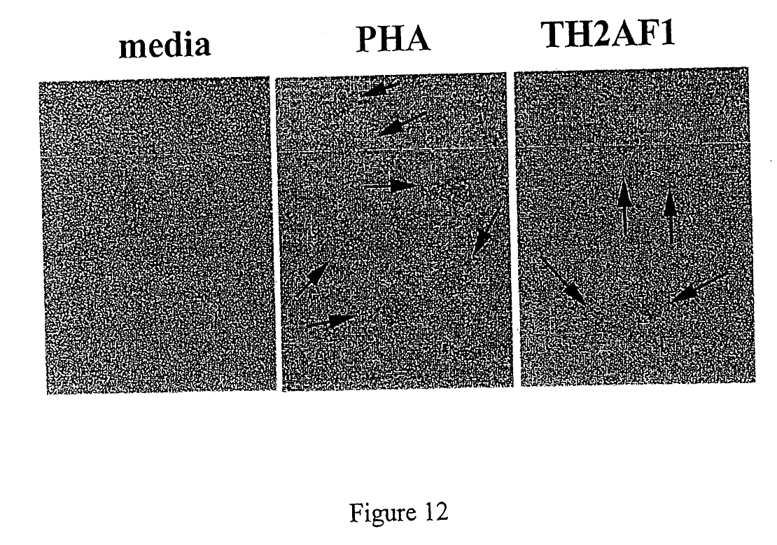

TH2AF1 can stimulate the mitogenic activity of mouse and human lymphocytes (as shown in Example 13) when produced by mammalian cells. The recombinant production of TH2AF1 can also be achieved using other recombinant expression systems such as Pichia and e. coli. To demonstrate this, the full-length murine TH2AF1 was cloned into the methanol-inducible pPIC yeast expression vector (Invitrogen). Briefly, the cDNA was digested from a cloning vector using an XhoI site inserted at codon 18 of the full-length cDNA and XbaI, located downstream of the natural stop codon. The insert was purified and cloned into the XhoI-XbaI site in the pPIC vector. The XhoI site allows for an in-frame fusion to occur with the α-factor contained within the pPIC vector for secretion from yeast. The recombinant vector was then transfected into Pichia following the manufacturer's protocol (Invitrogen). Recombinant yeast clones were screened by western blot for TH2AF1 expression using the antisera described in Example 7. A recombinant clone A1 was found to produce secreted TH2AF1 upon methanol induction. Aliquots of the supernatant from this clone were collected at various times after methanol induction and added to 25 μl of protein sample buffer for analysis on an 18% Tris-glycine SDS gel (Novex). Western blots were carried out using anti-TH2AF1 antiserum as described (Nicolaides et al., (1997) Proc. Natl. Acad. Sci. USA 94, 13175-13180). The result shown in FIG. 11 demonstrates the production of secreted TH2AF1 from clone A1 and that maximal expression of TH2AF1 occurs forty-eight hours after methanol induction. The supernatant from this clone and a pPIC empty vector clone induced with methanol for forty-eight hours were then used to measure mitogenic-activity of murine splenocytes. Briefly, spleens were removed from (B6D2)F1 as described (Nicolaides et al., (1997) Proc. Natl. Acad. Sci. USA 94, 13175-13180). Isolated splenocytes were then washed and plated at 1×10⁵ cell/ml and 0.1 ml was aliquoted into ninety-six well microtiter plates supplemented with 5 μg/ml PHA or 10% conditioned medium for TH2AF1 or empty vector producing Pichia. As shown in FIG. 12, splenocytes were activated by the addition of TH2AF1. These data demonstrate the ability to produce biologically active TH2AF1 from yeast vectors for functional studies and to produce reagents for antisera generation.

### Example 15

### Expression of TH2AF1 in Asthmatics

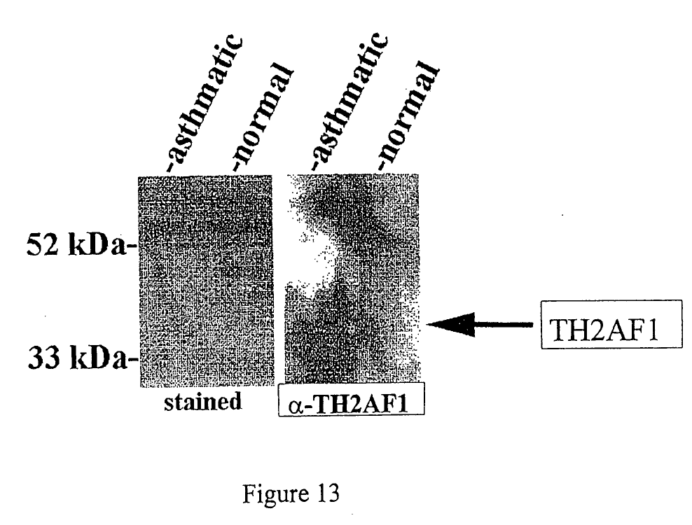

In mouse asthma models, TH2AF1 appears to be tightly associated with the asthmatic-like phenotype. To determine if TH2AF1 is produced in patients with clinically diagnosed asthma, the antisera described in Example 7 was used to screen bronchial alveolar lavage (BAL) samples from patients with or without asthma. As demonstrated in Example 12, TH2AF1 is a secreted molecule and if expressed by human lung cells should be secreted in the BAL fluid (BALF). Briefly, volunteer patients who were clinically diagnosed to have mild-grade asthma (free of steroid use) or normal individuals were given local anesthetics and their airways were lavaged using saline solution as described (Hunninghake et al., (1979) Am. J. Pathol. 97, 149-160). Recovered samples were then centrifuged at 2000 RPM to remove debris. For protein analysis, 50 μl of BALF was resuspended in protein sample buffer, boiled and samples were analyzed by Western blot as described in Example 14. FIG. 13 shows a representation of the data, where BALF from asthmatic patients has detectable levels of TH2AF1 in the airways in contrast to patients that are not asthmatic. This data demonstrates that TH2 AF1 is associated with human asthma and may therefore represent a pharmaceutical target to block the disease. In addition, this data suggests that TH2AF1 may serve as a diagnostic marker for the identification of low-grade asthma.

While the invention has been described and illustrated herein by references to various specific materials, procedures and examples, it is understood that the invention is not restricted to the particular combinations of material and procedures selected for that purpose. Numerous variations of such details can be implied as will be appreciated by those skilled in the art. It is intended that the specification and examples be considered as exemplary, only, with the true scope and spirit of the invention being indicated by the following claims. All references, patents and patent applications referred to in this application are herein incorporated by reference in their entirety.

1. An isolated nucleic acid molecule selected from the group consisting of:
(a) an isolated nucleic acid molecule that encodes the amino acid sequence of SEQ ID NO: 2, 4, 6, 8, 10 or 12;
(b) an isolated nucleic acid molecule that encodes a fragment of at least six (6) amino acids of SEQ ID NO: 2, 4, 6, 8, 10 or 12;
(c) an isolated nucleic acid molecule which hybridizes to a nucleic acid molecule comprising the complement of SEQ ID NO: 1, 3, 5, 7, 9 or 11 under conditions of sufficient stringency to produce a clear signal;
(d) an isolated nucleic acid molecule which hybridizes to the complement of a nucleic acid molecule that encodes the amino acid sequence of SEQ ID NO: 2, 4, 6, 8, 10 or 12 under conditions of sufficient stringency to produce a clear signal; and
(e) an isolated nucleic acid molecule with about seventy two (72) percent sequence homology to SEQ ID NO: 1, 3, 5, 7, 9 or 11.

2-9. (canceled)

10. A method for producing a polypeptide comprising the step of culturing a host cell transformed with the nucleic acid molecule of claim 1 under conditions in which the protein encoded by said nucleic acid molecule is expressed.

11. The method of claim 10, wherein said host cell is selected from the group consisting of prokaryotic hosts and eukaryotic hosts.

12. An isolated polypeptide produced by the method of claim 11.

13. An isolated polypeptide selected from the group consisting of an isolated protein comprising the amino acid sequence of SEQ ID NO: 2, 4, 6, 8, 10, or 12; an isolated polypeptide comprising a fragment of at least six amino acids of SEQ ID NO: 2, 4, 6, 8, 10, or 12; an isolated polypeptide comprising conservative amino acid substitutions of SEQ ID NO: 2, 4, 6, 8, 10, or 12; and naturally occurring amino acid sequence variants or isoforms of SEQ ID NO: 2, 4, 6, 8, 10, or 12.

14. An isolated antibody that binds to a polypeptide of claim 13.

15. The antibody of claim 14 wherein the antibody is produced from peptides comprising the ligand binding sequences of SEQ ID NO: 12.

16. The antibody of claim 14 wherein said antibody is a monoclonal or polyclonal antibody.

17. (canceled)

18. A method of treating asthma and asthma-related disorders in a mammal comprising the step of administering an effective amount of an agent which modulates at least one activity of a protein comprising the sequence of SEQ ID NO: 12.

19. The method of claim 17 wherein the expression is down-regulated.

20. The method of claim 18 wherein the activity is decreased.

21-36. (canceled)

37. An isolated antibody that binds to a polypeptide of claim 12.


SEQUENCE LISTING (27 sequences)

SEQ ID NO: 1 - DNA, 1104 bp, Mus musculus
  CDS (67)..(1005): mTH2AF1 sequence from FVB & TG5 lung
gaaaggttcctgtcattactcagctagcaactctcagctcctgcctggtgcagagggaagaccaccatgacccaactgggcttcctgctgtttatcatggttgccaccagagggtgcagtgcagctgaagagaacctgaacaccaacagatggggcaaatatttttttttctctctgcccagaagctgcaaggaaatcaagcaggaggacacaaaggcacaagatggtctctatttcctgcgcacggagaatggtgtcatctaccagaccttctgtgacatgaccactgcaggtggtggctggaccctggtggctagtgtgcatgagaacaacatgcgtgggaagtgcactgtgggtgatcgctggtccagtcagcaaggcaacagagctgactacccagagggggatggcaactgggccaactacaacacctttgggtctgcagaggctgccacaagtgatgactacaagaaccctggctacttcgacatccaggctgagaacctgggcatctggcatgtgcccaacaaaagccccctgcacacctggaggaacagctccctgctgaggtaccgcaccttcactggcttcctgcagcacttgggacataacctgtttggcctctaccagaagtacccagtgaaatacaacgaaggaaagtgttggactgacaatggcccagcattacctgtggtctatgactttggtgatgctcagaagacagcctcttattactccccctatggccagatggaattcactgcaggatatgttcagttcagagtatttaataatgagagagcagccagtgccttgtgtgctggcatgaaggtcactggatgtaatactgaatatcactgcatcggtggaggaggattcttcccagaaggtaaccccgtgcagtgtggagactttgctgcatttgattggaatggatatggaactcacatttggtacagcagtagccgggagataactgaagcagctgtgcttctattctatcgctgagaactctgcgggattggccttgacttctccattgtgggctccaaggcatgagaaactctgacttagtaactagaatgctaatgagcaataaagcag

SEQ ID NO: 2 - protein, 313 aa, Mus musculus
MTQLGFLLFIMVATRGCSAAEENLNTNRWGKYFFFSLPRSCKEIKQEDTKAQDGLYFLRTENGVIYQTFCDMTTAGGGWTLVASVHENNMRGKCTVGDRWSSQQGNRADYPEGDGNWANYNTFGSAEAATSDDYKNPGYFDIQAENLGIWHVPNKSPLHTWRNSSLLRYRTFTGFLQHLGHNLFGLYQKYPVKYNEGKCWTDNGPALPVVYDFGDAQKTASYYSPYGQMEFTAGYVQFRVFNNERAASALCAGMKVTGCNTEYHCIGGGGFFPEGNPVQCGDFAAFDWNGYGTHIWYSSSREITEAAVLLFYR

SEQ ID NO: 3 - DNA, 1104 bp, Mus musculus
  CDS (67)..(1005): mTH2AF1 sequence from FVB small intestine
gaaaggttcctgtcattactcagctagcaactctcagctcctgcctggtgcagagggaagaccaccatgacccaactgggattcctgctgtttatcatggtggctaccagaggttgcagtgcagctgaagagaacctggacaccaacaggtggggcaattctttcttttcctctctgcccagaagctgcaaggaaatcaagcaggagcacacaaaggcacaagatggtctctatttcctgcgcacgaagaatggtgtcatctaccagaccttctgtgacatgaccactgcaggtggtggctggaccctggtggctagtgtgcatgagaacaacatgcgtgggaagtgcactgtgggtgatcgatggtccagtcagcaaggcaacagagctgactacccagagggggatggcaactgggccaactacaacacctttgggtctgcagaggctgccacaagtgatgactacaagaaccctggctactttgacatccaggctgagaacctgggcatctggcatgtgcccaacaaaagccccctgcacaactggaggaagagctccctgctgaggtaccgcaccttcactggcttcctgcagcacttgggacataatctgtttggcctctacaagaagtacccggtgaaatacggagaaggaaagtgttggactgacaatggtccagcattacctgtagtctatgactttggtgatgctcggaagacagcctcttattactccccctctggccagagggaatttactgcaggatatgttcagttcagagtgtttaataatgagagagcggccagtgccttgtgtgctggcgtgagggtcactggatgtaatactgaacatcactgcatcggtggaggaggattcttcccagaaggtaaccccgtgcagtgtggagactttgcttcatttgattgggatggatatggaactcacaatgggtacagcagtagccggaagataactgaagcagccgtgcttctgttttatcgctgagaactctgcgggattggccctgacttctccattgtgggctccaaggcatgagaaacactgacttagtaactgaaatgctaatgagcaataaagcag

SEQ ID NO: 4 - protein, 313 aa, Mus musculus
MTQLGFLLFIMVATRGCSAAEENLDTNRWGNSFFSSLPRSCKEIKQEHTKAQDGLYFLRTKNGVIYQTFCDMTTAGGGWTLVASVHENNMRGKCTVGDRWSSQQGNRADYPEGDGNWANYNTFGSAEAATSDDYKNPGYFDIQAENLGIWHVPNKSPLHNWRKSSLLRYRTFTGFLQHLGHNLFGLYKKYPVKYGEGKCWTDNGPALPVVYDFGDARKTASYYSPSGQREFTAGYVQFRVFNNERAASALCAGVRVTGCNTEHHCIGGGGFFPEGNPVQCGDFASFDWDGYGTHNGYSSSRKITEAAVLLFYR

SEQ ID NO: 5 - DNA, 1104 bp, Mus musculus
  CDS (67)..(1005): mTH2AF1 sequence from TG5 transgenic mouse small intestine
gaaaggttcctgtcattactcagctagcaactctcagctcctgccctgtgcagagggaagaccaccatgacccaactgggcttcctgctgtttatcatggttgccaccagagggtgcagtgcagctgaaaagaacctggacaccaacagatggggcaattctttcttttcctctctgcccaaaagctgcaaggaaatcaagcaggaggacagaaaggcacaagatggtctctatttcctgcgcacgaagaatggtgtcatctaccagaccttctgtgacatgaccactgcaggtggtggctggaccctggtggctagtgtgcacgagaacaacatgcacgggaagtgcactgtgggcgatcgctggtccagtcagcaaggcaacagagctgattacccagagggggatggcaactgggccaactacaacacctttgggtctgcagagggtgccacaagtgatgactacaagaaccctggctactttgacatccaggctgagaacctgggcatctggcatgtgcccaacaacagccccctgcacaactggaggaacagctccctgctgaggtaccgcaccttcactggcttcctgcagcgcttgggccataatctgtttggtctctaccagaagtatccggtgaaatatggagaaggaaagtgttggactgacaatggcccagcattacctgtggtctatgactttggtgatgctcagaagacagcctcttattactcaccctctggccggaatgaattcactgcaggatatgttcagttcagagtgttcaataatgagagagcagccagtgccttgtgtgctggcgtgagggtcactggatgtaatactgaacatcactgcatcggtggaggaggattcttcccagaaggtaaccccgtgcagtgtggagactttgcgtcatttgatgcgaatggatatggaactcacatttggtacagcaatagccgggagataactgaagcagctgtgcttctgttttatcgctgagaactctgcgggattggccctgacttctccattgtgggctccaaggcatgagaaccactgacatagtaactaaaatgctaatgagcaataaagcag

SEQ ID NO: 6 - protein, 313 aa, Mus musculus
MTQLGFLLFIMVATRGCSAAEKNLDTNRWGNSFFSSLPKSCKEIKQEDRKAQDGLYFLRTKNGVIYQTFCDMTTAGGGWTLVASVHENNMHGKCTVGDRWSSQQGNRADYPEGDGNWANYNTFGSAEGATSDDYKNPGYFDIQAENLGIWHVPNNSPLHNWRNSSLLRYRTFTGFLQRLGHNLFGLYQKYPVKYGEGKCWTDNGPALPVVYDFGDAQKTASYYSPSGRNEFTAGYVQFRVFNNERAASALCAGVRVTGCNTEHHCIGGGGFFPEGNPVQCGDFASFDANGYGTHIWYSNSREITEAAVLLFYR

SEQ ID NO: 7 - DNA, 1104 bp, Mus musculus
  CDS (67)..(1005): mTH2AF1 sequence from DBA2 small intestine
  unsure (1080): n = a or c or g or t.
gaaaggttcctgtcattactcagctagcaactctcagctcctgcctggtgcagagggaagaccaccatgacccaactgggattcctgctgtttatcatggtggctaccagaggttgcagtgcagctgaagagaacctggacaccaacaggtggggcaattctttcttttcctctctgcccagaagctgcaaggaaatcaagcaggagcacacaaaggcacaagatggtctctatttcctgcgcacgaagaatggtgtcatctaccagaccttctgtgacatgaccactgcaggtggtggctggaccctggtggctagtgtgcatgagaacaacatgcgtgggaagtgcactgtgggtgatcgatggtccagtcagcaaggcaacagagctgactacccagagggggatggcaactgggccaactacaacacctttgggtctgcagaggctgccacaagtgatgactacaagaaccctggctactttgacatccaggctgagaacctgggcatctggcatgtgcccaacaaaagccccctgcacaactggaggaagagctccctgctgaggtaccgcaccttcactggcttcctgcagcacttgggacataatctgtttggcctctacaagaagtacccggtgaaatacggagaaggaaagtgttggactgacaatggtccagcattacctgtagtctatgactttggtgatgctcggaagacagcctcttattactccccctctggccagagggaatttactgcaggatatgttcagttcagagtgtttaataatgagagagcggccagtgccttgtgtgctggcgtgagggtcactggatgtaatactgaacatcactgcatcggtggaggaggattcttcccagaaggtaaccccgtgcagtgtggagactttgcttcatttgattgggatggatatggaactcacaatgggtacagcagtagccggaagataactgaagcagccgtgcttctgttttatcgctgagaactctgcgggattggccctgacttctccattgtgggctccaaggcatgagaaacactgacttagtaactngaatgctaatgagcaataaagcag

SEQ ID NO: 8 - protein, 313 aa, Mus musculus; identical to SEQ ID NO: 4

SEQ ID NO: 9 - DNA, 1104 bp, Mus musculus
  CDS (67)..(1005): mTH2AF1 sequence from C57BL/6 small intestine
  unsure (1081): r = a or g.
gaaaggttcctgtcattactcagctagcaactctcagctcctgcctggtgcagagggaagaccaccatgacccaactgggattcctgctgtttatcatggttgctaccagaggttgcagtgcagctgaagagaacctggacaccaacaggtggggcaattctttcttttcctctctgcccagaagctgcaaggaaatcaagcaggagcacacaaaggcacaagatggtctctatttcctgcgcacgaagaatggtgtcatctaccagaccttctgtgacatgaccactgcaggtggtggctggaccctggtggctagtgtgcatgagaacaacatgcgtgggaagtgcactgtgggtgatcgctggtccagtcagcaaggcaacagagctgactacccagagggggatggcaactgggccaactacaacacctttgggtctgcagaggctgccacaagtgatgactacaagaaccctggctactttgacatccaggctgagaacctgggcatctggcacgtgcccaacaaaagccccctgcacaactggaggaagagctccctgctgaggtaccgcaccttcactggcttcctgcagcacttgggacataatctgtttggcctctacaagaagtacccagtgaaatacggagaaggaaagtgttggactgacaatggcccagcattacctgtggtctatgactttggtgatgctcggaagacagcctcttattactccccctctggccagagggaatttactgcaggatatgttcagttcagagtgtttaataatgagagagcggccagtgccttgtgtgctggcgtgagggtcactggatgtaatactgaacatcactgcatcggtggaggaggattcttcccagaaggtaaccccgtgcagtgtggagactttgcgtcatttgattgggatggatatggaactcacaatgggtacagcagtagccggaagataactgaagcagctgtgcttctgttttatcgctgagaactctgcgggattggccctgacttctccattgtgggctccaaggcatgagaaacactgacttagtaactgraatgctaatgagcaataaagcag

SEQ ID NO: 10 - protein, 313 aa, Mus musculus; identical to SEQ ID NO: 4

SEQ ID NO: 11 - DNA, 1092 bp, Homo sapiens
  CDS (104)..(1042): hTH2AF1 sequence from A549 human cell line
tgaaaasctgcactctgttgagctccagggcgcagtggagggagggagtgaaggagctctctgtacccaaggaaagtgcagctgagactcagacaagattacaatgaaccaactcagcttcctgctgtttctcatagcgaccaccagaggatggagtacagatgaggctaatacttacttcaaggaatggacctgttcttcgtctccatctctgcccagaagctgcaaggaaatcaaagacgaatgtcctagtgcatttgatggcctgtattttctccgcactgagaatggtgttatctaccagaccttctgtgacatgacctctgggggtggcggctggaccctggtggccagcgtgcatgagaatgacatgcgtgggaagtgcacggtgggcgatcgctggtccagtcagcagggcagcaaagcagactacccagagggggacggcaactgggccaactacaacacctttggatctgcagaggcggccacgagcgatgactacaagaaccctggctactacgacatccaggccaaggacctgggcatctggcacgtgcccaataagtcccccatgcagcactggagaaacagctccctgctgaggtaccgcacggacactggcttcctccagacactgggacataatctgtttggcatctaccagaaatatccagtgaaatatggagaaggaaagtgttggactgacaacggcccggtgatccctgtggtctatgattttggcgacgcccagaaaacagcatcttattactcaccctatggccagcgggaattcactgcgggatttgttcagttcagggtatttaataacgagagagcagccaacgccttgtgtgctggaatgagggtcaccggatgtaacactgagcaccactgcattggtggaggaggatactttccagaggccagtccccagcagtgtggagatttttctggttttgattggagtggatatggaactcatgttggttacagcagcagccgtgagataactgaggcagctgtgcttctattctatcgttgagagttttgtgggagggaacccagacctctcctcccaaccatgagatc

SEQ ID NO: 12 - protein, 313 aa, Homo sapiens
MNQLSFLLFLIATTRGWSTDEANTYFKEWTCSSSPSLPRSCKEIKDECPSAFDGLYFLRTENGVIYQTFCDMTSGGGGWTLVASVHENDMRGKCTVGDRWSSQQGSKADYPEGDGNWANYNTFGSAEAATSDDYKNPGYYDIQAKDLGIWHVPNKSPMQHWRNSSLLRYRTDTGFLQTLGHNLFGIYQKYPVKYGEGKCWTDNGPVIPVVYDFGDAQKTASYYSPYGQREFTAGFVQFRVFNNERAANALCAGMRVTGCNTEHHCIGGGGYFPEASPQQCGDFSGFDWSGYGTHVGYSSSREITEAAVLLFYR

SEQ ID NO: 13 - DNA, 28 bp, Artificial Sequence
  Description of Artificial Sequence PCR antisense primer mLectin7
gggttcttgtagtcatcacttgtggcag

SEQ ID NO: 14 - DNA, 27 bp, Artificial Sequence
  Description of Artificial Sequence PCR antisense primer mLectin9
tgcagacccaaaggtgttgtagttggc

SEQ ID NO: 15 - DNA, 28 bp, Artificial Sequence
  Description of Artificial Sequence PCR antisense primer mLectin10
ctgccacaagtgatgactacaagaaccc

SEQ ID NO: 16 - DNA, 25 bp, Artificial Sequence
  Description of Artificial Sequence PCR antisense primer mLectin8
cgtgcagtgtggagactttgctgca

SEQ ID NO: 17 - DNA, 24 bp, Artificial Sequence
  Description of Artificial Sequene PCR sense primer mLectin20
gaaaggttcctgtcattactcagc

SEQ ID NO: 18 - DNA, 24 bp, Artificial Sequence
  Description of Artificial Sequence PCR antisense primer mLectin23
ctgctttattgctcattagcattc

SEQ ID NO: 19 - DNA, 26 bp, Artificial Sequence
  Description of Artificial Sequence PCR antisense primer hLectin7
agggttcttgtagtcatcgctcgtgg

SEQ ID NO: 20 - DNA, 26 bp, Artificial Sequence
  Description of Artificial Sequence PCR antisense primer hLectin9
agatccaaaggtgttgtagttggccc

SEQ ID NO: 21 - DNA, 28 bp, Artificial Sequence
  Description of Artificial Sequence PCR antisense primer hLectin2
gctctagatctcatggttgggaggaggg

SEQ ID NO: 22 - DNA, 21 bp, Artificial Sequence
  Description of Artificial Sequene PCR sense primer hLectin16
gaaagctgcactctgttgagc

SEQ ID NO: 23 - DNA, 20 bp, Artificial Sequence
  Description of Artificial Sequene PCR sense primer hLectin18
gcagctgagactcagacaag

SEQ ID NO: 24 - DNA, 55 bp, Artificial Sequence
  Description of Artificial Sequence  M use sense PCR primer
ggatcctaatacgactcactatagggagaccaccatggcagctgaagagaacctg

SEQ ID NO: 25 - DNA, 51 bp, Artificial Sequence
  Description of Artificial Sequence Mouse antisense PCR primer
ggtccttatcacttgtcatcgtcgtccttgtagtcgcgatagaatagaagc

SEQ ID NO: 26 - DNA, 61 bp, Artificial Sequence
  Description of Artificial Sequence Human sense PCR primer
ggatcctaatacgactcactatagggagaccaccatgagtacagatgaggctaatacttac

SEQ ID NO: 27 - DNA, 57 bp, Artificial Sequence
  Description of Artificial Sequence Human antisense PCR primer
ggatccttatcacttgtcatcgtcgtccttgtagtcacgatagaatagaagcacagc